(19) United States
(12) Patent Application Publication
(10) Pub. No.: US 2021/0353769 A1
(43) Pub. Date: Nov. 18, 2021

(54) SURFACE MODIFIED EXTRACELLULAR VESICLES

(71) Applicant: City University of Hong Kong, Kowloon (HK)

(72) Inventors: Jiahai Shi, Kowloon (HK); Thi Nguyet Minh Le, Kowloon (HK); Likun Wei, Kowloon (HK); Chanh Tin Pham, Kowloon (HK); Waqas Muhammad Usman, Kowloon (HK); Migara Kavishka Jayasinghe, Kowloon (HK)

(73) Assignee: City University of Hong Kong, Kowloon (HK)

(21) Appl. No.: 17/278,280

(22) PCT Filed: Sep. 20, 2019
(86) PCT No.: PCT/SG2019/050481

§ 371 Date: Mar. 19, 2021

Related U.S. Application Data
(60) Provisional application No. 62/776,009, filed on Dec. 6, 2018.
(60) Provisional application No. 62/734,303, filed on Sep. 21, 2018.

(51) Int. Cl. A61K 47/69; A61K 47/68

(52) U.S. Cl. CPC A61K 47/6901; A61K 47/6835

(57) ABSTRACT

The invention relates to surface modified extracellular vesicles, wherein the extracellular vesicles comprise an exogenous polypeptide tag that is covalently linked to a membrane protein of the extracellular vesicles. In a particular embodiment, the tag is covalently linked to the membrane protein of microvesicles by sortase-mediated ligation. Methods of preparing said extracellular vesicles and methods of using said extracellular vesicles loaded with therapeutic molecules for treating a disease are also disclosed herein.

### FIELD OF THE INVENTION

The present invention relates to extracellular vesicles and particularly, although not exclusively, to surface modified extracellular vesicles.

### BACKGROUND

RNA therapeutics including small-interfering RNAs (siRNAs), microRNAs (miRNAs), antisense oligonucleotides (ASOs), messenger RNAs (mRNAs), long non-coding RNAs and CRISPR-Cas9 genome editing guide RNAs (gRNAs) are emerging modalities for programmable therapies that target the diseased human genome with high specificity and flexibility. Common vehicles for RNA drug delivery, including viruses (e.g., adenoviruses, adeno-associated viruses, lentiviruses, retroviruses), lipid transfection reagents, and lipid nanoparticles, are usually immunogenic and/or cytotoxic. Thus a safe and effective strategy for the delivery of RNA drugs to most primary tissues and cancer cells, including leukemia cells and solid tumor cells, remains elusive.

Extracellular vesicles (EVs) have been applied to deliver RNA to patients. EVs are secreted by all types of cells in the body for intercellular communication. EVs comprised of exosomes which are small vesicles (10-100 nm) derived from the multivesicular bodies, microvesicles (100-1000 nm) derived from the plasma membrane of live cells and apoptotic bodies (500-5000 nm) derived from plasma membrane of apoptotic cells. EV-based drug delivery methods are desired but EV production has limitations. To produce highly pure and homogenous EVs, stringent purification methods such as sucrose density gradient ultracentrifugation or size exclusion chromatography are needed but they are time-consuming and not scalable. Moreover the yield is so low that billions of cells are needed to get sufficient EVs, and such numbers of primary cells are usually not available. Immortalization of primary cells would run the risk of transferring oncogenic DNA and retrotransposon elements along with the RNA drugs. In fact, all nucleated cells present some level of risk for horizontal gene transfer, because it is not predictable a priori which cells already harbor dangerous DNA, and which do not. Accordingly, there remains a need for effective approach for delivering nucleic acid material to patients with reduced side effects.

Further, to make EV-based therapy more specific, EVs may be engineered to have peptides or antibodies that bind specifically to certain target cell, by expressing peptides or antibodies in donor cells from plasmids that are transfected or transduced using retrovirus or lentivirus followed by an antibiotic based or fluorescence-based selection. These methods pose a high risk of horizontal gene transfer as the highly expressed plasmids are likely incorporated into EVs and eventually transferred to the target cells. Genetic elements in the plasmids may cause oncogenesis. If stable cell lines are made to produce EVs, abundant oncogenic factors including mutant DNAs, RNAs and proteins are packed in EVs and deliver to the target cells the risk of tumorigenesis. On the other hand, genetic engineering methods are not applicable to red blood cells as plasmids cannot be transcribed in red blood cells because of the lack of ribosomes. It is also not applicable to stem cells and primary cells that are hard to transfect or transduce.

Recently, there is a new method of coating EVs with antibodies fused to a C1C2 domain of lactadherin that bind to phosphatidylserine (PS) on the surface of EVs. This method allows conjugation of EVs with antibodies without any genetic modification. However, C1C2 is a hydrophobic protein and hence requires a tedious purification method in mammalian cells and storage in bovine serum albumin containing buffer. Moreover, the conjugation of EVs with C1C2-fusion antibodies is based on the affinity binding between C1C2 and PS that is transient.

Accordingly, there remains a strong need for a stable EV for therapeutic or diagnostic purpose. The present invention has been devised in light of the above considerations.

Methods for Sortase-mediated functionalization of M13 bacteriophage capsid proteins have been previously presented (US 2014/0030697 A1). This functionalization enables a variety of structures on the surface of viruses, and is useful for creating new viral surface modifications that can be exploited for the creation of surface interactions.

A method of sortagging for surface modification of red blood cells has previously been developed through the use of genetically engineered cells (WO 2014/183071 A2), In this method, human CD34+ progenitor cells are genetically engineered to express a fusion protein comprising a red blood cell membrane protein and a peptide of interest. In some embodiments, the fusion protein comprises a type II red blood cell transmembrane protein fused to a peptide comprising a sequence recognized by a sortase for surface modification.

The conjugation of agents to mammalian cells has previously been seen WO 2014/183066 A2). This document presents methods of conjugating agents to mammalian cells through the contacting a living cell with a sortase and a sortase substrate comprising a sortase recognition motif and an agent in the presence of a sortase.

### SUMMARY OF THE INVENTION

The inventors have devised a method for enzymatic modification of the surface of extracellular vesicles. Accordingly, this disclosure relates to modified extracellular vesicles comprising, on their surface, a tag, as well as methods for making and using such modified extracellular vesicles.

Extracellular vesicles (EVs) are emerging drug delivery vehicles due to their natural biocompatibility, high delivery efficiency, low toxicity, and low immunogenic characteristics. EVs are usually engineered by genetic modifications of their donor cells however, genetic engineering methods are inefficient in primary cells and eventually post a risk of horizontal gene transfer which is unsafe for clinical applications. Here, we describe a method to modify the surface of EVs using protein ligase enzymes for covalent conjugation of molecules including peptides, small molecules, proteins and antibodies. This method is simple, safe and efficient for EV engineering. It can be applied for many types of EVs including those from primary cells. The extracellular vesicle is a membrane-derived vesicle, and thus comprises a membrane, normally a lipid bilayer.

EV-mediated delivery of drugs including small molecules, proteins and nucleic acids is an attractive platform because of the natural biocompatibility of EVs that overcome most in vivo delivery hurdles. EVs are generally nontoxic and non-immunogenic. They are taken up readily by many cell types but they may possess some antiphagocytic markers such as CD47 that help them to evade the phagocytosis by macrophages and monocytes of the reticuloendothelial system. Moreover, EVs are able to extravasate well through the interendothelial junctions and even cross the blood-brain barrier hence, they are greatly versatile drug carriers.³ Of clinical value, delivery by EVs is not hampered by the multidrug resistance mechanism caused by overexpression of P-glycoproteins that tumor cells often use to eliminate many chemical compounds.

At its most general, this disclosure provides an extracellular vesicle having, on its surface, a tag. The tag may be a peptide, polypeptide or protein. The tag is preferably exogenous, meaning that it is not normally found on the surface of the extracellular vesicles. The tag may be covalently linked to the extracellular vesicle. For example, the tag may be covalently linked to the membrane of the extracellular vesicle. It may be linked to a protein within the membrane of the extracellular vesicle, such as a protein with an N-terminal glycine or with residues having side chain amino groups, such as Asparagine, Glutamine, Arginine, Lysine and Histidine. The peptide, polypeptide or protein may be conjugated with a small molecule such as biotin, a FLAG epitope (FLAG tag), HA-tag, or polyhistidine (e.g. a 6×His tag). In some cases, the tag may comprise one or more of biotin, a FLAG tag, an HA-tag, or a polyhistidine.

These may facilitate detection, isolation or purification of the tag. The peptide or small molecule is optionally a ligand that is bound by a receptor on the surface of a target cell. The peptide, polypeptide or protein may be a targeting moiety or a binding moiety. In some cases, the targeting moiety or binding moiety is an antibody or antigen binding fragment. In some aspects, the antigen binding fragment is a single domain antibody (sdAb) or a single chain antibody (scAb). The sdAb/scAb may have binding affinity for a target cell. The tag may comprise a therapeutic molecule or entity. The tag may comprise a labelling molecule or entity.

The extracellular vesicle may be a microvesicle or an exosome. Although the extracellular vesicle may be derived from any suitable cell, extracellular vesicles derived from red blood cells (RBCs) are particularly contemplated herein.

In certain aspects, the extracellular vesicles described herein are loaded with a cargo or a plurality of cargo molecules. In other words, the extracellular vesicles encapsulate a cargo, such as a protein, peptide, small molecule or nucleic acid. The cargo may be loaded endogenously or exogenously. The cargo may be therapeutic. The cargo may be Paclitaxel. The cargo may be a labelling molecule or entity, such as a detectable small molecule. In some cases, the cargo is a nucleic acid selected from the group consisting of an antisense oligonucleotide, an siRNA, a miRNA, an mRNA, a gRNA or a plasmid. The cargo may be exogenous, meaning that it is not normally found within the cell from which the extracellular vesicles are derived.

Also disclosed herein are compositions comprising one or more extracellular vesicles as disclosed herein. Preferably, at least 75%, at least 80%, at least 85%, at least 90%, at least 95%, at least 97% or substantially all of the extracellular vesicles in the composition are linked to the tag. In some cases, at least 75%, at least 80%, at least 85%, at least 90%, at least 95%, at least 97% or substantially all of the extracellular vesicles in the composition encapsulate a cargo or a plurality of cargo molecules.

Also disclosed herein are extracellular vesicles and compositions containing extracellular vesicles used in medicine. Such compositions and extracellular vesicles may be administered in an effective amount to a subject in need of treatment. The subject may be in need of treatment for, or may have, a genetic disorder, inflammatory disease, cancer, autoimmune disorder, cardiovascular disease or a gastrointestinal disease. The cancer optionally selected from leukemia, lymphoma, myeloma, breast cancer, lung cancer, liver cancer, colorectal cancer, nasopharyngeal cancer, kidney cancer or glioma.

In certain aspects disclosed herein, there is provided a tagged extracellular vesicle obtained by a method comprising: obtaining an extracellular vesicle and linking the extracellular vesicle to a tag. The tag is preferably linked by a covalent bond. It may be linked to a molecule in the membrane of the extracellular vesicle, such as a molecule at the surface of the membrane. The extracellular vesicle may be linked to the tag using a protein ligase.

In a further aspect, there is provided a method of inhibiting the growth or proliferation of a cancer cell comprising contacting the cancer cell with an extracellular vesicle or composition according to the invention. Also disclosed herein are in vitro methods comprising contacting a cell with an extracellular vesicle.

Methods of producing modified extracellular vesicles are also disclosed herein, as well as extracellular vesicles obtained by such methods. At its most general, such methods involve contacting an extracellular vesicle with a tag and a protein ligase under conditions which allow covalent binding between the tag and a surface protein of the extracellular vesicle. Such methods may also involve a step of contacting the extracellular vesicle with a cargo and electroporating to encapsulate the cargo with the extracellular vesicle. The extracellular vesicle may be contacted with the cargo before or after contacting with the tag. Preferably, the extracellular vesicle is contacted with the cargo prior to contacting with the tag. In that case, the extracellular vesicle that is contacted with the tag and the protein ligase is an extracellular vesicle that encapsulates a cargo molecule, or a “loaded extracellular vesicle”. Methods of producing modified extracellular vesicles may further include a step of purifying, isolating or washing the extracellular vesicle. Such a step will occur after the extracellular vesicle has been tagged with the tag. Purifying, isolating or washing the extracellular vesicle may involve differential centrifugation of the extracellular vesicle. Differential centrifugation may involve centrifugation in a sucrose gradient, or a frozen sucrose cushion. In preferred aspects, the protein ligase used to covalently link the tag to the extracellular vesicle is a sortase or asparaginyl endopeptidases (AEP) and their derivatives. Preferably, the ligase is sortase A or a derivative thereof. The ligase may be asparaginyl endopeptidase 1 or a derivative thereof. The ligase is preferably washed from the extracellular vesicles or otherwise removed, after the tag has been linked to the extracellular vesicle.

Methods described herein utilise a tag. The tag will comprise a protein ligase recognition sequence. The protein ligase recognition sequence will be selected to correspond to the ligase used to tag the extracellular vesicles. For example, where the ligase is a sortase, the tag will comprise a sortase recognition sequence. The tag may optionally comprise a spacer or linker. The spacer or linker is preferably arranged between the binding molecule and the peptide recognition site of the tag. The spacer may be a flexible linker, for example a peptide linker comprising around 10 or more amino acids.

The linker peptide may have a ligase-binding site at the C-terminus that allows it to be conjugated to EVs using a peptide ligase and a reactive amino acid residues (such as GL) at the N-terminus that allows it to react to the peptide ligase for conjugation to a sdAb.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1.Conjugation of EVs with a single domain antibody (sdAb) using sortase enzyme. A. Experimental workflow for purification of sortase A, sdAb and conjugation of EVs. B. Gel electrophoresis analysis of proteins before (input) and after (elutant) FPLC purification of His-tagged sortase A (18 kDa). C. Gel electrophoresis analysis of proteins before (input) and after (elutant) FPLC purification of anti-mCherry (mC) sdAb variable heavy chain (VHH) with His tag, Myc tag, HA tag, FLAG tag and sortase binding site LPETG (20 kDa in total). D. Average concentration and size distribution of RBCEVs from 3 donors with the SEM in grey (100,000×dilution). E. Representative transmission electron microscopy images of RBCEVs at 86000× (right) magnification. Scale bar, 200 nm. F. Western blot (VVB) analysis of His tag in sortase A, sdAb and RBCEVs before and after the sortagging reaction.

FIG. 2.Conjugation of EVs with a peptide using sortase enzyme. A. Western Blot (WB) analysis of biotin on RBCEVs conjugated with a peptide containing one part of CD47 for self recognition or “don't eat me” signal, a sortase binding sequence, and a biotin tag. B. Western blot analysis of biotin on RBCEVs from 3 different donors, purified separately and conjugated with YG20 peptide containing an EGFR-binding sequence, a sortase-binding sequence and a biotin tag (bi-YG20), using sortase A reaction. Biotin was detected using HRP-conjugated streptavidin. C. FACS analysis of Alexa-Fluor-647 (AF647, APC channel) versus forward scatter (FSC-A) in AF647-conjugated streptavidin beads (Strep-AF647) binding to uncoated RBCEVs or to RBCEVs coated with bi-YG20 using sortase A reaction. D. The most abundant proteins in RBCEVs identified using mass spectrometry (score >1,000). Protein interactions were predicted based on known interactions in RBCs. E. Membrane proteins sortagged to biotinylated peptides identified using biotin-streptavidin pulldown and mass spectrometry. Mass spectrometry (MS) score was calculated based on the abundance and detection confidence.

FIG. 3. Uptake of YG20-peptide-coated EVs by EGFR-positive SKBR3 breast cancer cells. A. FACS analysis of PKH26 (PE channel) versus FSC-A in SKBR3 cells with low or high EGFR expression, treated with uncoated RBC-EVs or YG20 peptide-coated RBCEVs, gated as in B. All RBCEVs were labelled with PKH26. The supernatant from the last wash of the RBCEV labelling experiment was used as a negative control. B. Gating EGFR low and EGFR high SKBR3 populations. EGFR expression was detected using anti-EGFR antibody conjugated with FITC. C. Average percentage of PKH26-positive cells determined in A, ±SEM (n=3 repeats). Student's t-test results are shown as **P<0.01.

FIG. 4. Conjugation of EVs with a peptide using sortase enzyme. A. Gel electrophoresis of the OaAPE1(49.7 kDa) and His-Ub-OaAPE1 ligase (59.5 kDa with His-Ub tag) after affinity purification and SEC purification from E.coli transformed with the OaAEP1 expression vector. B. Western blot analysis of biotin on RBCEVs conjugated with a biotinylated TRNGL peptide using OaAEP1 ligase, detected by HRP-conjugated streptavidin. C. FACS analysis of Alexa-Fluor-647 (AF647, APC channel) versus forward scatter area (FSC-A) in AF647-conjugated streptavidin beads (Strep-AF647) binding to uncoated RBCEVs or to RBCEVs coated with bi-TRNGL using OaAEP1 ligase. D. Western blot analysis of biotin in RBCEVs conjugated with biotinylated EGFR-targeting (ET) peptide using ligase. E. Comparison of biotin detection in bi-TR-peptide-ligated RBCEVs from 3 different donors (D1-D3) with a serial dilution of biotinylated horseradish peroxidase (HRP). The number of peptides per EV was calculated based on the intensity of the Western blot bands relative to copies of biotinylated HRP in the serial dilution.

FIG. 5. Approaches for specific delivery. RBCEVs are conjugated with sdAb or peptide using protein ligases such as sortase A or OaAEP1 then loaded with therapeutic drugs such as cytotoxic small molecules, RNAs, DNAs for gene therapies, proteins for therapies or diagnosis. The peptide and sdAbs bind to specific receptors on the surface of the target cells leading to the delivery of the drugs by RBCEVs and subsequent therapeutic effects in the target cells.

FIG. 6. The addition of a tag to an extracellular vesicle. This schematic demonstrates a representative example of how a tag with a protein ligase recognition sequence (LPETG in this representative example) can be added to the extracellular vesicle through the action of a protein ligase (Sortase A in this representative example).

FIG. 7. Ligation of leukemia EVs with peptides. A. Size exclusion chromatography purification of EVs from THP1 cells, eluted in 30 fractions. EVs were detected using a Nanosight particle analyser and protein concentration was measured using a BCA assay. B. Western blot analysis of biotin in THP1 EVs conjugated with biotinylated TRNGL peptide using OaAEP ligase.

FIG. 8. Specific binding of EGFR-targeting peptide promotes the uptake of sortagged RBCEVs by EGFR-positive cells. (A) Expression of EGFR in human leukaemia (MOLM13), breast cancer (SKBR3 and CA1a) and lung cancer (H358 and HCC827) cells, analysed using FACS with a FITC anti-EGFR antibody. (B) Binding of biotinylated control (Cont) or EGFR-targeting (ET) peptide to 3 indicated cell lines, shown by a FACS analysis of biotin-bound AF647-streptavidin. (C) FACS analysis of Calcein AM fluorescence in H358 cells treated with RBCEVs that were labelled with Calcein-AM and conjugated with Cont or ET-peptides using sortase A. Colours in the histogram is presented in the same pattern as in the graph. Student's t-test ***P<0.001.

FIG. 9. Ligase-mediated conjugation of RBCEVs with EGFR-targeting peptides also enhances the specific uptake of RBCEVs. (A) FACS analysis of Calcein AM fluorescence in H358 cells treated with Calcein-AM labelled RBCEVs that were conjugated with Cont or ET-peptides using OaEAP1 ligase. (B) Effect of blocking peptides, which compete for binding to EGFR, on the uptake of ligated RBCEVs. (C) Effect of chemical inhibitors, EIPA (blocking macropinocytosis), Filipin (blocking clathrin-mediated endocytosis), Wortmannin (blocking mannose-receptor-mediated endocytosis), on the uptake of RBCEVs that were labeled with Calcein-AM-labeled and conjugated with ET peptide. Student's t-test *P<0.05, ***P<0.001.

FIG. 10. EGFR-targeting RBCEVs are enriched in the lung of mice bearing EGFR-positive lung cancer. (A) (A) Conditioning the mice with the ghost membrane of RBCs or with intact RBCs was performed by retro-orbital injection of the ghost or RBCs 1 hour before injection of DiR-labelled RBCEVs in the tail vein. After 24 hours, fluorescence was observed in the organs. (B) NSG mice were injected with 1 million H358-luciferase cells in the tail vein. After 3 weeks, bioluminescence were detected in the lung using the in vivo imaging system (IVIS). Mice with lung cancer were preconditioned with RBCs by retro-orbital injection. After 1 hour, the mice were injected with 0.1 mg DiR-labeled RBCEVs. After 8 hours, DiR fluorescence were observed in the organ using the IVIS. Representative images of mice with lung cancer shown by bioluminescent signals in the lung 3 weeks after i.v. injection of H358-luciferase cells. Representative DiR fluorescent images the organs from the mice injected with uncoated RBCEVs, control/ET peptide-ligated RBCEVs or with the flowthrough of the RBCEV wash. Mean DiR fluorescent intensity in each organ relative to the average mean intensity, subtracted by signals detected in the flow-through controls. Student's t-test *P<0.05, **P<0.001.

FIG. 11. Conjugation with a self-peptide prevent phagocytosis of RBCEVs and enhance the availability of RBCEVs in the circulation. (A) FACS analysis of Calcein AM in MOLM13 and THP1 monocytes that are treated with control or self peptide (SP) ligated RBCEVs. Colours in the histogram is presented the same as in the graphs. (B) FACS analysis of streptavidin beads bound by biotinylated anti-GPA antibody that captured RBCEVs in the plasma of NSG mice 5 minutes after an injection of 0.2 mg CFSE-labelled RBCEVs in the tail vein. (C) Biodistribution of DiR-labeled RBCEVs that were conjugated with a control peptide or SP using sortase A. Student's t-test ***P<0.001.

FIG. 12. Conjugation of RBCEVs with sdAbs is enhanced with a linker peptide. (A) Gel electrophoresis analysis of EGFR VHH sdAbs before (input) and after (elutant) a His-tag affinity purification. (B) Schema of a 2-step ligation reaction. In the first step, a linker peptide with ligase-binding site is conjugated to proteins with GL on RBCEVs. In the second step, the linker peptide is ligated to the VHH with NGL. (C) Western blot analysis of VHH (using an anti-VHH antibody) on RBCEVs conjugated with EGFR-targeting VHH using OaAEP1 ligase. Ligated RBCEVs were washed with SEC. (D) FACS analysis of GPA (RBCEV marker) on uncoated RBCEVs or ET-VHH ligated RBCEVs that bound to HCC827 cells at 4oC after a 1-hour incubation.

FIG. 13. Single-domain antibodies promote specific uptake of RBCEVs by target cells. (A) FACS analysis of Calcein AM in EGFR-positive H358 cells treated with RBCEVs that were labelled with calcein AM and conjugated with EGFR-targeting VHH sdAb with or without the linker peptide using OaAEP1 ligase. (B) FACS analysis of Calcein AM in CA1a cells expressing surface mCherry, treated with RBCEVs that were labelled with calcein AM and conjugated with mCherry-targeting VHH sdAb with or without the linker peptide using OaAEP1 ligase. Colours are presented the same in the histograms and the bar graphs. Student's t-test *P<0.05, **P<0.05, ***P<0.001.

FIG. 14. Delivery of RNAs and drugs using EGFR-targeting RBCEVs. (A) Delivery of luciferase mRNA to H358 cells using ET-VHH-ligated RBCEVs. Luciferase activity was measured in the lysate of H358 cells after 24 hours of treatment. Uncoated and mCherry-VHH-ligated RBCEVs were included as negative controls. (B) Delivery schema of paclitaxel (PTX) to H358 tumors using RBCEVs. PTX was loaded into RBCEVs using sonication. Loaded RBCEVs were washed and ligated with ET peptide. NSG mice were injected with H358 cells in the tail vein. After 3 weeks when the tumor was detected in the lung, the mice were treated with RBCEVs or PTX only every 3 days for 5 times. Bioluminescence was also measured every 3 days. (C) Loading efficiency of PTX in RBCEVs, determined using HPLC. (D) Bioluminescent signals in the upper body of mice treated with 20 mg/kg PTX only, or an equivalent dose of PTX loaded in RBCEVs with or without EGRF-targeting peptide every 3 days. The bioluminescence was measured in the lung area every 3 days from the first day of treatments using the IVIS. A representative image of the mouse in each condition is shown.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

Described herein is a method of modifying a surface of an extracellular vesicle with a tag in the presence of an enzyme such as a protein or protein ligase or variant. The tag may be a binding molecule that allows the extracellular vesicle to bind to a target cell for target specific delivery.

Extracellular Vesicles

The term “extracellular vesicle” as used herein refers to a small vesicle-like structure released from a cell into the extracellular environment.

Extracellular vesicles (EVs) are substantially spherical fragments of plasma membrane or endosomal membrane between 50 and 1000 nm in diameter. Extracellular vesicles are released from various cell types under both pathological and physiological conditions. Extracellular vesicles have a membrane. The membrane may be a double layer membrane (i.e. a lipid bilayer). The membrane may originate from the plasma membrane. Accordingly, the membrane of the extracellular vesicle may have a similar composition to the cell from which it is derived. In some aspects disclosed herein, the extracellular vesicles are substantially transparent.

The term extracellular vesicles encompasses exosomes, microvesicles, membrane microparticles, ectosomes, blebs and apoptotic bodies. Extracellular vesicles may be produced via outward budding and fission. The production may be a natural process, or a chemically induced or enhanced process. In some aspects disclosed herein, the extracellular vesicle is a microvesicle produced via chemical induction.

Extracellular vesicles may be classified as exosomes, microvesicles or apoptotic bodies, based on their size and origin of formation. Microvesicles are a particularly preferred class of extracellular vesicle according to the invention disclosed herein. Preferably, the extracellular vesicles of the invention have been shed from the plasma membrane, and do not originate from the endosomal system.

Extracellular vesicles disclosed herein may be derived from various cells, such as red blood cells, white blood cells, cancer cells, stem cells, dendritic cells, macrophages and the like. In a preferred example, the extracellular vesicles are derived from a red blood cell, although extracellular vesicles from any source may be used, such as from leukemia cells and cell lines.

Microvesicles or microparticles arise through direct outward budding and fission of the plasma membrane. Microvesicles are typically larger than exosomes, having diameters ranging from 100-500 nm. In some cases, a composition of microvesicles comprises microvesicles with diameters ranging from 50-1000 nm, from 50-750 nm, from 50-500 nm, from 50-300 nm, from 101-1000 nm, from 101-750 nm, from 101-500 nm, or from 100-300 nm, or from 101-300 nm. Preferably, the diameters are from 100-300 nm. A population of microvesicles, for example as present in a composition, pharmaceutical composition, medicament or preparation, will comprise microvesicles with a range of different diameters, the median diameter of microvesicles within a microvesicle sample can range from 50-1000 nm, from 50-750 nm, from 50-500, from 50-300 nm, from 101-1000 nm, from 101-750 nm, from 101-500 nm, or from 100-300 nm, or from 101-300 nm. Preferably, the median diameter is between 100-300 nm.

The diameter of exosomes range from around 30 to around 100 nm. In some cases, a composition of exosomes comprises exosomes with diameters ranging from 10-200 nm, from 10-150 nm, from 10-120 nm, from 10-100 nm, from 20-150 nm, from 20-120 nm, from 25-110 nm, from 25-100 nm, or from 30-100 nm. Preferably, the diameters are from 30-100 nm. A population of exosomes, for example as present in a composition, pharmaceutical composition, medicament or preparation, will comprise exosomes with a range of different diameters, the median diameter of exosomes within a sample can range ranging from 10-200 nm, from 10-150 nm, from 10-120 nm, from 10-100 nm, from 20-150 nm, from 20-120 nm, from 25-110 nm, from 25-100 nm, or from 30-100 nm. Preferably, the median diameter is between 30-100 nm.

Exosomes are observed in a variety of cultured cells including lymphocytes, dendritic cells, cytotoxic T cells, mast cells, neurons, oligodendrocytes, Schwann cells, and intestinal epithelial cells. Exosomes originate from the endosomal network that locates in within mutivesicular bodies, large sacs in the cytoplasm. These sacs fuse to the plasma membrane, before being released into extracellular environment.

Apoptotic bodies or blebs are the largest extracellular vesicle, ranging from 1-5 μm. Nucleated cells undergoing apoptosis pass through several stages, beginning with condensation of the nuclear chromatin, membrane blebbing and finally release of EVs including apoptotic bodies.

Preferably, the extracellular vesicles are derived from human cells, or cells of human origin. The extracellular vesicles of the invention may have been induced from cells contacted with a vesicle inducing agent. The vesicle inducing agent may be calcium ionophore, lysophosphatidic acid (LPA), or phorbol-12-myristat-13-acetate (PMA).

Red Blood Cell Extracellular Vesicles (RBC-EVs)

In certain aspects disclosed herein, the extracellular vesicles are derived from red blood cells. Red blood cells are a good source of EVs for a number of reasons. Because red blood cells are enucleated, RBC-EVs contain less nucleic acid than EVs from other sources. RBC-EVs do not contain endogenous DNA. RBC-EVs may contain miRNA or other RNAs. RBC-EVs are free from oncogenic substances such as oncogenic DNA or DNA mutations.

RBC-EVs may comprise haemoglobin and/or stomatin and/or flotillin-2. They may be red in colour. Typically RBC-EVs exhibit a domed (concave) surface, or “cup shape” under transmission electron microscopes. The RBC-EV may be characterised by having cell surface CD235a. RBC-EVs according to the invention may be about 100 to about 300 nm in diameter. In some cases, a composition of RBC-EVs comprises RBC-EVs with diameters ranging from 50-1000 nm, from 50-750 nm, from 50-500 nm, from 50-300 nm, from 101-1000 nm, from 101-750 nm, from 101-500 nm, or from 100-300 nm, or from 101-300 nm.

Preferably, the diameters are from 100-300 nm. A population of RBC-EVs will comprise RBC-EVs with a range of different diameters, the median diameter of RBC-EVs within a RBC-EV sample can range from 50-1000 nm, from 50-750 nm, from 50-500 nm, from 50-300 nm, from 101-1000 nm, from 101-750 nm, from 101-500 nm, or from 100-300 nm, or from 101-300 nm. Preferably, the median diameter is between 100-300 nm.

Preferably, the RBC-EVs are derived from a human or animal blood sample or red blood cells derived from primary cells or immobilized red blood cell lines. The blood cells may be type matched to the patient to be treated, and thus the blood cells may be Group A, Group B, Group AB, Group O or Blood Group Oh. Preferably the blood is Group O. The blood may be rhesus positive or rhesus negative. In some cases, the blood is Group O and/or rhesus negative, such as Type O−. The blood may have been determined to be free from disease or disorder, such as free from HIV, sickle cell anaemia, malaria. However, any blood type may be used. In some cases, the RBC-EVs are autologous and derived from a blood sample obtained from the patient to be treated. In some cases, the RBC-EVs are allogenic and not derived from a blood sample obtained from the patient to be treated.

RBC-EVs may be isolated from a sample of red blood cells. Protocols for obtaining EVs from red blood cells are known in the art, for example in Danesh et al. (2014) Blood. 2014 Jan. 30; 123(5): 687-696. Methods useful for obtaining EVs may include the step of providing or obtaining a sample comprising red blood cells, inducing the red blood cells to produce extracellular vesicles, and isolating the extracellular vesicles. The sample may be a whole blood sample. Preferably, cells other than red blood cells have been removed from the sample, such that the cellular component of the sample is red blood cells.

The red blood cells in the sample may be concentrated, or partitioned from other components of a whole blood sample, such as white blood cells. Red blood cells may be concentrated by centrifugation. The sample may be subjected to leukocyte reduction.

The sample comprising red blood cells may comprise substantially only red blood cells. Extracellular vesicles may be induced from the red blood cells by contacting the red blood cells with a vesicle inducing agent. The vesicle inducing agent may be calcium ionophore, lysophosphatidic acid (LPA), or phorbol-12-myristat-13-acetate (PMA).

RBC-EVs may be isolated by centrifugation (with or without ultracentrifugation), precipitation, filtration processes such as tangential flow filtration, or size exclusion chromatography. In this way, RBC-EVs may be separated from RBCs and other components of the mixture.

Extracellular vesicles may be obtained from red blood cells by a method comprising: obtaining a sample of red blood cells; contacting the red blood cells with a vesicle inducing agent; and isolating the induced extracellular vesicles.

The red blood cells may be separated from a whole blood sample containing white blood cells and plasma by low speed centrifugation and using leukodepletion filters. In some cases, the red blood cell sample contains no other cell types, such as white blood cells. In other words, the red blood cell sample consists substantially of red blood cells. The red blood cells may be diluted in buffer such as PBS prior to contacting with the vesicle inducing agent. The vesicle inducing agent may be calcium ionophore, lysophosphatidic acid (LPA) or phorbol-12-myristat-13-acetate (PMA). The vesicle inducing agent may be about 10 nM calcium ionophore. The red blood cells may be contacted with the vesicle inducing agent overnight, or for at least 1, at least 2, at least 3, at least 4, at least 5, at least 6, at least 7, at least 8, at least 9, at least 10, at least 11, at least 12 or more than 12 hours. The mixture may be subjected to low speed centrifugation to remove RBCs, cell debris, or other non-RBC-EVs matter and/or passing the supernatant through an about 0.45 um syringe filter. RBC-EVs may be concentrated by ultracentrifugation, such as centrifugation at around 100,000 ×g. The RBC-EVs may be concentrated by ultracentrifugation for at least 10 minutes, at least 20 minutes, at least 30 minutes, at least 40 minutes, at least 50 minutes or at least one hour. The concentrated RBC-EVs may be suspended in cold PBS. They may be layered on a 60% sucrose cushion. The sucrose cushion may comprise frozen 60% sucrose.

The RBC-EVs layered on the sucrose cushion may be subject to ultracentrugation at 100,000×g for at least one hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours or more. Preferably, the RBC-EVs layered on the sucrose cushion may be subject to ultracentrugation at 100,000×g for about 16 hours. The red layer above the sucrose cushion is then collected, thereby obtaining RBC-EVs. The obtained RBC-EVs may be subject to further processing, such as washing, tagging, and optionally loading.

Tap

The extracellular vesicles according to the invention have, at their surface, a tag. The tag is preferably a protein or peptide sequence. The tag may be a peptide or protein. It may be a modified peptide or protein, such as a glycosylated or biotinylated protein or peptide. The tag may be covalently linked to the extracellular vesicle, such as covalently linked to a membrane protein in the extracellular vesicle. The tag may have been added to the extracellular vesicle after the extracellular vesicle had formed. The tag may be linked to the extracellular vesicle by a sequence that comprises or consists of a sequence that is, or that is derived from, a protein ligase recognition sequence. For example, the tag may be linked to the extracellular vesicle by a sequence that comprises 100% sequence identity to a protein ligase recognition sequence, or about 90%, about 80%, about 70%, about 60%, about 50% or about 40% sequence identity to a protein ligase recognition sequence. The amino acid sequence may comprises LPXT.

The tag is presented on the external surface of the vesicle, and is thus exposed to the extravesicular environment. The inventors have found that surface modification of the extracellular vesicles reduces the uptake of the extracellular vesicle by macrophage and improves the availability of extracellular vesicles in the circulation, as well as enhancing the specific delivery of non-endogenous substances or cargos to the target cells.

The tag may be an exogenous molecule. In other words, the tag is a molecule that is not present on the external surface of the vesicle in nature. In some cases, the tag is an exogenous molecule that is not present in the cell or red blood cell from which the extracellular vesicle is derived.

The tag may increase the stability, uptake efficiency and availability in the circulation of the extracellular vesicles. Such tags may enhance the effects of extracellular vesicles that have already some intrinsic therapeutic properties such as extracellular vesicles from mesenchymal stem cells or from dendritic cells for cardiac regeneration or vaccination respectively.

In some cases, the tag acts to present the extracellular vesicles and extracellular vesicles containing cargos in the circulation and organs in the body. The peptides and proteins can act as therapeutic molecules such as blocking/activating target cell function or presenting antigens for vaccination. They can also act as probes for biomarker detection such as diagnosis of toxins.

The tag preferably contains a functional domain and a protein ligase recognition sequence. The functional domain may be capable of binding to a target moiety, capable of detection, or capable of inducing a therapeutic effect. The functional domain may be capable of binding to a target molecule. Tags comprising such a functional domain may be referred to herein as binding molecules. A binding molecule is one that is capable of interacting specifically with a target molecule. Extracellular vesicles comprising a binding moiety may be particularly useful for delivering a cargo or a therapeutic agent to a cell that has the target molecule. Suitable binding molecules include antibodies and antigen binding fragments (sometimes known as antibody fragments), ligand molecules and receptor molecules. The binding molecule will bind to a target of interest. The target may be a molecule associated with, such as expressed on the surface of, a cell of interest, such as a cancer cell. The ligand may form a complex with a biomolecule on the target cell, such as a receptor molecule.

Suitable binding molecules include antibodies and antigen binding fragments. Fragments, such as Fab and Fab₂ fragments may be used as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855). Antibodies or antigen binding fragments useful in the extracellular vesicles disclosed herein will recognise and/or bind to, a target molecule. The target molecule may be a protein present on the surface of a cancer cell.

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299. Antibodies and fragments useful herein may be human or humanized, murine, camelid, chimeric, or from any other suitable source.

By “ScFv molecules” we mean molecules wherein the VH and VL partner domains are covalently linked, e.g. directly, by a peptide or by a flexible oligopeptide. Fab, Fv, ScFv and sdAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and sdAb fragments are monovalent, having only one antigen combining site. Monovalent antibody fragments are particularly useful as tags, because of their small size.

A preferred binding molecule for use herein is a sdAb. By “sdAb” we mean single domain antibody consisting of one, two or more single monomeric variable antibody domains. sdAb molecules are sometimes referred to as dAb.

In some cases, the binding molecule is a single chain antibody, or scAb. A scAb consists of covalently linked VH and VL partner domains (e.g. directly, by a peptide, or by a flexible oligopeptide) and optionally a light chain constant domain.

Other suitable binding molecules include ligands and receptors that have affinity for a target molecule. The tag may be a ligand of a cell surface receptor, such as an EGFR binding peptide. Examples include streptavidin and biotin, avidin and biotin, or ligands of other receptors, such as fibronectin and integrin.

The small size of biotin results in little to no effect to the biological activity of bound molecules. As biotin and streptavidin, biotin and avidin, and fibronectin and integrin bind their pairs with high affinity and specificity, they are very useful as binding molecules. The Avidin-biotin complex is the strongest known non-covalent interaction (Kd=10-15 M) between a protein and ligand. Bond formation is rapid, and once formed, is unaffected by extremes of pH, temperature, organic solvents and other denaturing agents. The binding of biotin to streptavidin and is also strong, rapid to form and useful in biotechnology applications.

The functional domain may comprise or consist of a therapeutic agent. The therapeutic agent may be an enzyme. It may be an apoptotic inducer or inhibitor.

The functional domain may comprise an antigen, antibody recognition sequence or T cell recognition sequence. The tag may comprise one or more short peptides derived from one or more antigenic peptides. The peptide may be a fragment of an antigenic peptide. Suitable antigenic peptides are known to one of skill in the art.

The functional domain may comprise or consist of a detectable moiety. Detectable moieties include fluorescent labels, colorimetric labels, photochromic compounds, magnetic particles or other chemical labels. The detectable moiety may be biotin or a His tag.

The tag may comprise a spacer or linker moiety. The spacer or linker may be arranged between the tag and the protein ligase recognition sequence. The spacer or linker may be linked to the N or C terminus of the tag. Preferably the spacer or linker is arranged so as not to interfere or impede the function of the tag, such as the target binding activity by the tag. The spacer or linker is preferably a peptide sequence. In particular aspects, the spacer or linker is a series of at least 1, at least 2, at least 3, at least 4, at least 5, at least 6, at least 7, at least 8, at least 9, at least 10 amino acids, at least 11 amino acids, at least 12 amino acids, at least 13 amino acids, at least 14 amino acids or at least 15 amino acids. Preferably, the spacer or linker is flexible. The spacer may comprise a plurality of glycine and/or serine amino acids.

Spacer and linker sequences are known to the skilled person, and are described, for example in Chen et al., Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen et al., Adv Drug Deliv Rev (2013) 65(10): 1357-1369. Flexible linker sequences often comprise high proportions of glycine and/or serine residues.

In some embodiments, the spacer or linker sequence comprises at least one glycine residue and/or at least one serine residue. In some embodiments the linker sequence consists of glycine and serine residues. In some embodiments, the spacer or linker sequence has a length of 1-2, 1-3, 1-4, 1-5 or 1-10 amino acids.

We observed that inclusion of the spacer or linker may improve the efficiency of the protein ligase reaction between the extracellular vesicle and the tag moiety. The term “tag” as used herein may encompass a peptide comprising a tag, a spacer, and protein ligase recognition sequence.

Suitable protein ligase recognition sequences are known in the art. The protein ligase recognition sequence is recognised by the protein ligase used in the method of tagging the extracellular vesicles. For example, if the protein ligase used in the method is a sortase, then the protein ligase recognition sequence is a sortase binding site. In those cases, the sequence may be LPXTG (where X is any naturally occurring amino acid), preferably LPETG. Alternatively, where the enzyme is AEP1, the protein ligase recognition sequence may be NGL. The protein ligase binding site may be arranged at the C terminus of the peptide or protein.

The tag may additionally comprise one or more further sequences to aid in purification or processing of the tag, during production of the tag itself, during the tagging method, or for subsequent purification. Any suitable sequence known in the art may be used. For example, the sequence may be an HA tag, a FLAG tag, a Myc tag, a His tag (such as a poly His tag, or a 6×His tag).

Provided herein is a method of producing a tag suitable for tagging an extracellular vesicle. The method may involve engineering a peptide. The method may involve chemically synthesising a peptide. The method may involve engineering a nucleic acid sequence to express the tag. For example, the method may involve preparing a nucleic acid construct encoding the tag. The nucleic acid construct may encode a polypeptide comprising the tag and one or more of a spacer sequence, a protein ligase recognition sequence, one or more further sequences. For example, the nucleic acid construct may encode a polypeptide comprising or consisting of a tag, a spacer and a protein ligase sequence.

Also provided is a nucleic acid encoding a tag as disclosed herein. The nucleic acid may be comprised within a vector. The vector may comprise nucleic acid encoding the tag, spacer and protein ligase recognition sequence. The vector may be an E.coli expression vector.

Tapping Method

Disclosed herein are methods of tagging an extracellular vesicle. The methods involve linking a tag to the surface of an extracellular vesicle. The methods may involve binding the tag to the extracellular vesicle, such as through a covalent bond. The methods may involve linking a tag to the membrane of the extracellular vesicle. Preferably, the tagging method disclosed herein does not involve C1C2 domain of lactadherin which is known to bind to phosphatidylserine (PS). Preferably, the tag is added to the extracellular vesicle after the vesicle has formed, rather than added to the cell from which the vesicle is derived, such that it is included in the vesicle during its formation. The method may comprise the step of contacting an extracellular vesicle and a tag with a protein ligase or its variant, and incubating the mixture under conditions which allow covalent binding between the tag and a surface protein of the extracellular vesicle. The conditions allow cleavage and joining of the tag to the surface of the extracellular vesicle. The conditions used depend on the ligase used.

In some methods disclosed herein, the extracellular vesicle is tagged with the tag in a single step process. In other words, the tag is prepared and ligated to the extracellular vesicle.

In other methods, the extracellular vesicle is tagged with the tag in a multi step process. In such methods, the extracellular vesicle is first ligated to a peptide to generate a peptide tagged extracellular vesicle, and then the peptide tagged extracellular vesicle is ligated to a functional domain such as a binding moiety or targeting moiety. In some methods, the extracellular vesicle is tagged to one or more peptides, prior to ligation with the functional domains. The method may involve contacting an extracellular vesicle with a peptide and first protein ligase under conditions which allow covalent binding between the peptide and a surface protein of the extracellular vesicle, thereby generating a peptide tagged extracellular vesicle. The method may then involve contacting the peptide tagged extracellular vesicle with a functional domain peptide and a second protein ligase under conditions which allow covalent binding between the peptide covalently bound to the extracellular vesicle and the functional domain peptide.

In these cases, the peptide may comprise a ligase binding site at either end of the peptide. The ligase binding sites may comprise a ligase recognition site and a ligase acceptor site. The peptide may comprise a ligase recognition site at one end, and a ligase acceptor site at the other end. Alternatively, the ligase binding sites may both comprise ligase recognition sites. The ligase recognition site may a specific site recognised by the ligase. The ligase may catalyse formation of a bond between one or more amino acid resides of the ligase recognition site and the ligase acceptor site. For example, the ligase recognition site may comprise NGL, and the ligase acceptor site may comprise GL.

The ligase binding sites may correspond to the same or different ligases. For example, the ligase binding sites may both be sortase binding sites, or may both be AEP1 binding sites. Alternatively, the ligase binding sites may correspond to different ligases, such as a sortase binding site and an AEP1 binding site. The first protein ligase may be the same ligase as the second protein ligase, or the first and second protein ligase may be different. In some cases, the first and second protein ligases are sortases. In some cases, the first and second protein ligases are both Sortase A.

The functional domain peptide may comprise one or more functional domains and a ligase binding site. The ligase binding site may comprise a ligase recognition site or a ligase acceptor site. Preferably the ligase binding site comprises a ligase recognition site. The ligase binding site corresponds to the ligase binding site on the peptide, such that the ligase may catalyse linkage between the ligase binding site of the peptide and the ligase binding site of the functional domain peptide.

The peptide and the functional domain peptide may comprise one more functional molecule sequences such as a biotin, a FLAG tag, HA-tag, His-tag or other sequence. Such methods may involve building the tag on the extracellular vesicle, with different components added in series, such as the linker, one or more functional domain such as detectable tags, binding moieties or targeting moieties.

In some cases, the method involves preparing each component separately. The method may involve preparing or providing extracellular vesicles, tags, linkers, peptides and/or ligase. The method may involve combining one, two or three components selected from the tag, the extracellular vesicle and the ligase to form a mixture. The mixture may contain further agents, such as a buffer. The mixture may be prepared by combining the components in any order. For example, the three components may be combined substantially simultaneously, or a mixture of two of the components may be prepared and stored for a time, prior to addition of the third agent.

The mixture may be incubated at about 0° C. to about 30° C., from about 4° C. to about 25° C., about 4° C. or about 25° C. for at least 15 minutes, 30 minutes, 1 hour, or 2 hours, or 3 hours. Preferably, the mixture is gently agitated. In this way, the protein ligase attaches the binding molecule on the surface of the extracellular vesicle by forming covalent bonding between the binding molecule and the surface protein of the extracellular vesicle.

Preferably, the pH of the mixture is acidic. The pH may be 8.0 or lower. The pH may be lower than 8, 7, 6, 5, 4, 3, 2 or 1.

The method may involve a step of isolating the modified extracellular vesicle from the mixture. The isolation may involve ultracentrifugation, or size exclusion chromatography or filtration. The differential centrifugation may include adding the resultant mixture to a frozen sucrose cushion and performing centrifugation. The term “sucrose cushion” refers to a sucrose gradient which establishes itself during centrifugation. The sucrose gradient may be prepared by using a solution of about 40% to about 70%, from about 50% to about 60% or about 60%, preferably about 60% sucrose.

In some cases, the tagged extracellular vesicle may be isolated by virtue of the tag, for example, by affinity chromatography. The isolation may utilise one or more functional domains of the tag peptide, such as the HA-tag, FLAG-tag, His-tag or other sequence.

After centrifugation, the purified modified extracellular vesicle is collected and optionally washed with a buffer solution such as phosphate-buffered saline (PBS). Centrifugation is then carried out to collect the purified modified extracellular vesicle. The method may comprise one or more washing steps. Preferably, the method comprises two or three washing steps.

The extracellular vesicle may be loaded or unloaded. In other words, the extracellular vesicle may encapsulate a cargo or comprise no exogenous material. In some cases, following linkage of the tag, the extracellular vesicle is loaded with a cargo. Preferably, the cargo is loaded following linkage of the tag. In other words, tagged extracellular vesicles are prepared. Cargo is then loaded into the tagged extracellular vesicles.

Preferred methods involve contacting an extracellular vesicle with a tag. The methods may involve further contacting the extracellular vesicle and the tag with a protein ligase. The extracellular vesicle and the tag may be contacted under conditions suitable for inducing the tag to link to the extracellular vesicle.

For example, the tag and vesicle may be contacted in a buffer, such as a protein ligase buffer. The vesicle and tag may be contacted for sufficient time for tagging to occur.

The method may involve the step of washing the tagged extracellular vesicles to remove ligase.

Also disclosed herein are extracellular vesicles that have, at their surface, a tag, said extracellular vesicles obtained by a method disclosed herein. Extracellular vesicles tagged in this way are different to extracellular vesicles which are obtained from tagged cells, and thus are tagged ab initio. For example, the linkage between the extracellular vesicle and the tag may be compositionally different.

In one embodiment, the method links a tag to the surface of the extracellular vesicle. The method may link a tag to the membrane of the extracellular vesicle. In one embodiment, the method links a tag to the surface of the extracellular vesicle through a covalent bond. In another embodiment, the method links a tag to the membrane of the extracellular vesicle through a covalent bond. In a further embodiment, the method of tagging an extracellular vesicle links an extracellular vesicle to a tag which contains a spacer or linker. In an additional embodiment, the method of tagging an extracellular vesicle links an extracellular vesicle to a tag which contains a functional molecule which is capable of being detected, or capable of inducing a therapeutic effect.

In one embodiment, the method of tagging an extracellular vesicle is performed under acidic conditions. In some embodiments, the method of tagging an extracellular vesicle includes the step of contacting an extracellular vesicle and a tag with a protein ligase. In some embodiments, the method of tagging an extracellular vesicle includes the step of contacting an extracellular vesicle and a tag with a sortase enzyme. In some embodiments, the method of tagging an extracellular vesicle includes the step of contacting an extracellular vesicle and a tag with Sortase A. In some embodiments, the method of tagging an extracellular vesicle tags an unloaded extracellular vesicle. In some embodiments, the method of tagging an extracellular vesicle tags a loaded extracellular vesicle.

Certain methods disclosed herein involve a step of formulating the tagged extracellular vesicles as a pharmaceutical product. This may involve the addition of one or more pharmaceutical excipients or carriers, such as buffers or preservatives. In some cases, the method may involve freezing, lyophilising or otherwise preserving the extracellular vesicles or composition comprising the extracellular vesicles.

Preparing the Tag

Methods disclosed herein may involve preparing the tag. The tag may be a recombinant protein. Preparation of the tag may involve molecular biology techniques such as those described in Sambrook et al., Molecular Cloning: A Laboratory Manual, New York: Cold Spring Harbor Press, 1989, or otherwise known in the art. The tag may have been prepared earlier and stored. For example, frozen, refrigerated, lyophilised or otherwise prepared earlier.

The tag must contain a binding site to enable binding to the EV. Thus, the tag is prepared or synthesized according to the type of protein ligase used, i.e. to include a corresponding binding site for the protein ligase to recognize. For example, where the protein ligase used is a sortase or its derivatives, the binding molecule bears a sortase binding site; or when the protein ligase is an AEP, like OaAEP1, the binding molecule bears an OaAEP1 binding site. Specifically, the Sortase A recognition sequence may be LPXTG (where X is any naturally occurring amino acid), preferably LPETG. The Sortase B recognition sequence may be NXZTN (where X is any naturally occurring amino acid) or NP(Q/K)(T/S)(N/G/S)(D/A), Sortase C enzymes demonstrate a unique variance in their ability to recognize a variety of sorting signals and amino groups.

The tag may be engineered to comprise a spacer or linker. The spacer or linker may be arranged between the binding molecule and the peptide recognition site of the tag. The spacer may be a flexible linker, for example a peptide linker comprising around 10 or more amino acids.

The linker peptide may be an independent peptide having a ligase-binding site (.e.g. NGL) at one end that allows it to be conjugated to EVs using a peptide ligase and reactive amino acid residues (such as GL) at the other end that allows it to react to the peptide ligase for conjugation to a sdAb. This linker peptide should be at least 10 amino acid. It may also contain a Myc tag, His tag or HA tag for detection purposes. It may contain a polyethylene glycol (PEG) to prevent phagocytosis.

To avoid oligomerization, 2 linker peptides can be used instead of 1. One linker peptide has a C-terminal NGL that allows its ligation to EVs and a cysteine conjugated with dibenzocyclooctyne (DBCO) group at the N-terminal. Another linker has an N-terminal GL that allows its ligation to a sdAb with NGL and a C-terminal Lys with azide group (N3). After 2 peptides are ligated separately to the RBCEVs and sdAbs, they can be connected using a click chemistry reaction between the DBCO and the azide group.

The tag may also optionally include a functional molecule capable of being detected, or capable of inducing a therapeutic effect. The functional molecule may be capable of binding to a target molecule. Extracellular vesicles comprising a functional molecule for binding may be particularly useful for delivering a cargo or a therapeutic agent to a cell that has the target molecule. Suitable functional binding molecules include antibodies and antigen binding fragments (sometimes known as antibody fragments), ligand molecules and receptor molecules. The binding molecule will bind to a target of interest. The target may be a molecule associated with, such as expressed on the surface of, a cell of interest, such as a cancer cell.

The functional domain may comprise or consist of a therapeutic agent. The therapeutic agent may be a small molecule, an enzyme or an apoptotic inducer or inhibitor.

The functional domain may comprise an antigen, antibody recognition sequence or T cell recognition sequence. The tag may comprise one or more short peptides derived from one or more antigenic peptides. The peptide may be a fragment of an antigenic peptide. Suitable antigenic peptides are known to one of skill in the art.

The functional domain may comprise or consist of a detectable moiety. Detectable moieties include fluorescent labels, colorimetric labels, photochromic compounds, magnetic particles or other chemical labels. The detectable moiety may be biotin or a His tag.

Preparation of the tag may comprise engineering a nucleic acid that encodes the tag. The nucleic acid may comprise a sequence encoding the functional domain and a protein ligase recognition sequence. The nucleic acid may also include nucleic acid encoding a spacer or linker. The nucleic acid encoding the spacer or linker may be arranged between the functional domain and the protein ligase recognition sequence.

A vector comprising nucleic acid encoding the tag is also provided. The vector may be an expression vector, for expression of the tag in a culture of cells, such as E. coli.

Protein Expression

Molecular biology techniques suitable for the producing peptides or polypeptides such as tags or cargo molecules according to the invention in cells are well known in the art, such as those set out in Sambrook et al., Molecular Cloning: A Laboratory Manual, New York: Cold Spring Harbor Press, 1989

The peptide may be expressed from a nucleotide sequence. The nucleotide sequence may be contained in a vector present in the cell, or may be incorporated into the genome of the cell.

A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express plant aspartic proteases from a vector according to the invention. Suitable vectors include plasmids, binary vectors, viral vectors and artificial chromosomes (e.g. yeast artificial chromosomes).

In this specification the term “operably linked” may include the situation where a selected nucleotide sequence and regulatory nucleotide sequence (e.g. promoter and/or enhancer) are covalently linked in such a way as to place the expression of the nucleotide sequence under the influence or control of the regulatory sequence (thereby forming an expression cassette). Thus a regulatory sequence is operably linked to the selected nucleotide sequence if the regulatory sequence is capable of effecting transcription of the nucleotide sequence. Where appropriate, the resulting transcript may then be translated into a desired protein or polypeptide.

Any cell suitable for the expression of polypeptides may be used for producing peptides according to the invention. The cell may be a prokaryote or eukaryote. Preferably the cell is a eukaryotic cell such as a yeast cell, a plant cell, insect cell or a mammalian cell. In some cases the cell is not a prokaryotic cell because some prokaryotic cells do not allow for the same post-translational modifications as eukaryotes.

In addition, very high expression levels are possible in eukaryotes and proteins can be easier to purify from eukaryotes using appropriate tags. Specific plasmids may also be utilised which enhance secretion of the protein into the media.

Methods of producing a peptide of interest such as a tag may involve culture or fermentation of a eukaryotic cell modified to express the peptide. The culture or fermentation may be performed in a bioreactor provided with an appropriate supply of nutrients, air/oxygen and/or growth factors. Secreted proteins can be collected by partitioning culture media/fermentation broth from the cells, extracting the protein content, and separating individual proteins to isolate secreted aspartic protease. Culture, fermentation and separation techniques are well known to those of skill in the art.

Bioreactors include one or more vessels in which cells may be cultured. Culture in the bioreactor may occur continuously, with a continuous flow of reactants into, and a continuous flow of cultured cells from, the reactor. Alternatively, the culture may occur in batches. The bioreactor monitors and controls environmental conditions such as pH, oxygen, flow rates into and out of, and agitation within the vessel such that optimum conditions are provided for the cells being cultured.

Following culture of cells that express peptide of interest, that peptide is preferably isolated. Any suitable method for separating proteins from cell culture known in the art may be used. In order to isolate a protein of interest from a culture, it may be necessary to first separate the cultured cells from media containing the protein of interest. If the protein of interest is secreted from the cells, the cells may be separated from the culture media that contains the secreted protein by centrifugation. If the protein of interest collects within the cell, for example in the vacuole of the cell, it will be necessary to disrupt the cells prior to centrifugation, for example using sonification, rapid freeze-thaw or osmotic lysis. Centrifugation will produce a pellet containing the cultured cells, or cell debris of the cultured cells, and a supernatant containing culture medium and the protein of interest.

It may then be desirable to isolate the protein of interest from the supernatant or culture medium, which may contain other protein and non-protein components. A common approach to separating protein components from a supernatant or culture medium is by precipitation. Proteins of different solubilities are precipitated at different concentrations of precipitating agent such as ammonium sulfate. For example, at low concentrations of precipitating agent, water soluble proteins are extracted. Thus, by adding different increasing concentrations of precipitating agent, proteins of different solubilities may be distinguished. Dialysis may be subsequently used to remove ammonium sulfate from the separated proteins.

Other methods for distinguishing different proteins are known in the art, for example ion exchange chromatography and size chromatography. These may be used as an alternative to precipitation, or may be performed subsequently to precipitation.

Peptides and proteins useful in the methods disclosed herein may be purified, or may have been subject to a purification step. The methods disclosed herein may involve one or more steps of purifying the proteins or peptides. For example, the protein or peptide may be purified using affinity chromatography.

Once the protein of interest has been isolated from culture it may be necessary to concentrate the protein. A number of methods for concentrating a protein of interest are known in the art, such as ultrafiltration or lyophilisation.

Protein Ligases

Tagging methods disclosed herein may involve the use of a protein ligase to link the extracellular vesicle to the tag. The protein ligase may be a transpeptidase. The terms protein ligase and peptide ligase are used interchangeably herein. Protein ligases suitable for use in the methods disclosed herein can be produced in large scale with high purity in bacteria such as E.coli at low cost. The ligase-mediated reactions are reproducible with predictable rates and targets. The ligase does not alter the physical properties of extracellular vesicles and ligase can be removed easily by washing.

Suitable protein ligases facilitate the incorporation of the tag on the surface of the extracellular vesicle. In other words, the tag acts as a substrate for the ligase.

Protein ligases used in the methods disclosed herein can be any enzyme capable of facilitating the joining of a substance to a protein by forming a chemical bond, preferably a covalent bond. In particular, protein ligases are capable of facilitating the joining of a tag to a molecule on or at the surface of an extracellular vesicle. Any variants of the protein ligase are also included in this invention such as, but not limited to, isozymes and alloenzymes. Variants having modification on the structure of the protein ligase without affecting the protein ligating effect are also included.

In some aspects, the protein ligase used to covalently link the tag to the extracellular vesicle is a sortase, a biotin protein ligase (BPL), a ubiquitin ligase, or asparaginyl endopeptidases (AEP) and their derivatives, such as AEP chimeric proteins, AEP fragments or AEP mutants. Preferably, the ligase is sortase A or a derivative thereof, such as sortase A chimeric proteins, sortase A fragments or sortase A mutants. The ligase may be asparaginyl endopeptidase 1 or a derivative thereof, such as asparaginyl endopeptidase 1 chimeric proteins, asparaginyl endopeptidase 1 fragments or asparaginyl endopeptidase 1 mutants. The ligase is preferably washed from the extracellular vesicles or otherwise removed, after the tag has been linked to the extracellular vesicle.

In some cases, the transpeptidase is a Sortase. Sortases are enzymes derived from prokaryotes that modify surface proteins by recognizing and cleaving a carboxyl terminal sorting signal. Sortases can link many peptides, all extended at their C-termini by a sortase recognition sequence, to unmodified proteins with N-terminal glycine residues on the RBC surface.

In some cases, the ligase is Sortase A, for example, Staphylococcus aureus Sortase A (NCBI accession: BBA25062.1 GI: 1236588748). Streptococcus pneumoniae Sortase A (NCBI accession: CTN13080.1 GI: 906766293), Listeria monocytogenes Sortase A (NCBI accession: KSZ47989.1 GI: 961372910), Enterococcus faecium Sortase A (NCBI accession: OZN21179.1 GI: 1234782246). Alternatively the ligase may be an enzyme with 100% sequence identity to a known Sortase A sequence, or about 90%, about 80%, about 70%, about 60%, about 50% or about 40% sequence identity to a known Sortase A sequence. Furthermore, the protein ligase may be an enzyme with the same enzymatic function as Sortase A.

In some cases, the ligase is Sortase B, for example, Staphylococcus aureus Sortase B (NCBI accession: KPE24466.1 GI: 929343259), Listeria monocytogenes Sortase B (NCBI accession: KSZ47109.1 GI: 961372026), Streptococcus pneumoniae Sortase B (NCBI accession: EJH14940.1 GI: 395904018), Clostridioides difficile Sortase B (NCBI accession: AKP43679.1 GI: 873321415). Alternatively the ligase may be an enzyme with 100% sequence identity to a known Sortase B sequence, or about 90%, about 80%, about 70%, about 60%, about 50% or about 40% sequence identity to a known Sortase B sequence. A sequence. Furthermore, the protein ligase may be an enzyme with the same enzymatic function as Sortase B

In some cases, the ligase is Sortase C, for example, Enterococcus faecium Sortase C (NCBI accession: KWW64427.1 GI: 984823861), Streptococcus pneumoniae Sortase C (NCBI accession: EIA07041.1 GI: 379642509), Bacillus cereus Sortase C (NCBI accession: AJG96560.1 GI: 753363636), Listeria monocytogenes Sortase B (NCBI accession: WP_075491524.1 GI: 1129540689). Alternatively the ligase may be an enzyme with 100% sequence identity to a known Sortase C sequence, or about 90%, about 80%, about 70%, about 60%, about 50% or about 40% sequence identity to a known Sortase C sequence. A sequence. Furthermore, the protein ligase may be an enzyme with the same enzymatic function as Sortase C.

Where the enzyme is a sortase, the method of tagging the extracellular vesicle is a Sortagging method. The Sortase A recognition sequence may be LPXTG (where X is any naturally occurring amino acid), preferably LPETG. The Sortase B recognition sequence may be NXZTN (where X is any naturally occurring amino acid), or may be NP(Q/K)(T/S)(N/G/S)(D/A), Sortase C enzymes demonstrate a unique variance in their ability to recognize a variety of sorting signals and amino groups.

In some cases, the protein ligase is AEP1 (asparaginyl endopeptidase 1). It may be Oldenlandia affinis OaAEP1 (NCBI Accession: ALG36105.1 GI: 931255808). It may be the OaAEP1-Cys247 Ala peptidase, or a variant thereof. It may also be an Arabidopsis thaliana asparaginyl endopeptidase (e.g. NCBI Accession: Q39119.2 GI: 148877260), an Oryza sativa asparaginyl endopeptidase (e.g. NCBI accession: BAC41387.1 GI: 26006022), a Clitoria ternatea asparaginyl endopeptidase (e.g. NCBI accession: ALL55653.1 GI: 944204395). Alternatively the ligase may be an enzyme with 100% sequence identity to a known asparaginyl endopeptidase sequence, or about 90%, about 80%, about 70%, about 60%, about 50% or about 40% sequence identity to a known asparaginyl endopeptidase sequence. A sequence. Furthermore, the protein ligase may be an enzyme with the same enzymatic function as an asparaginyl endopeptidase.

Where the enzyme is an asparaginyl endopeptidase, the protein ligase recognition sequence may be NGL.

In some cases, the protein ligase is a butelase. It may be Clitoria ternatea Butelase 1 (E.g. NCBI accession: 6DHI_A GI: 1474889693). Alternatively the ligase may be Clitoria ternatea Butelase 2

Protein ligases useful in the methods disclosed herein may be obtained from commercial sources, or may be generated in E. coli or other bacterial or yeast cell culture.

Cargo

Extracellular vesicles disclosed herein may be loaded with, or contain, a cargo. The cargo, also referred to as the load, may be a nucleic acid, peptide, protein, small molecule, sugar or lipid. The cargo may be a non-naturally occurring or synthetic molecule. The cargo may be a therapeutic molecule, such as a therapeutic oligonucleotide, peptide, small molecule, sugar or lipid. In some cases, the cargo is not a therapeutic molecule, for example a detectable moiety or visualization agent. The cargo may exert a therapeutic effect in the target cell after being delivered to that target cell. For example, the cargo may be a nucleic acid which is expressed in the target cell. It may act to inhibit or enhance the expression of a particular gene or protein of interest. For example, the protein or nucleic acid may be used to edit a target gene for gene silencing or modification.

Preferably, the cargo is an exogenous molecule, sometimes referred to as a “non-endogenous substance”. In other words, the cargo is a molecule that does not naturally occur in the extracellular vesicle, or the cell from which it is derived. Such a cargo is preferably loaded into the extracellular vesicles after the vesicles have formed, rather than loaded or produced by the cell, such that it is also contained within the extracellular vesicles.

In some cases, the cargo may be a nucleic acid. The cargo may be RNA or DNA. The nucleic acid may be single stranded or double stranded. The cargo may be an RNA. The RNA may be a therapeutic RNA. The RNA may be a small interfering RNA (siRNA), a messenger RNA (mRNA), a guide RNA (gRNA), a circular RNA, a microRNA (miRNA), a piwiRNA (piRNA), a transfer RNA (tRNA), or a long noncoding RNA (IncRNA) produced by chemical synthesis or in vitro transcription. In some cases, the cargo is an antisense oligonucleotide, for example, having a sequence that is complementary to an endogenous nucleic acid sequence such as a transcription factor, miRNA or other endogenous mRNA.

The cargo may be encode a molecule of interest. For example, the cargo may be an mRNA that encodes Cas9 or another nuclease.

In the cell, the antisense nucleic acids hybridize to the corresponding mRNA, forming a double-stranded molecule. The antisense nucleic acids interfere with the translation of the mRNA, since the cell will not translate an mRNA that is double-stranded. The use of antisense methods to inhibit the in vitro translation of genes is well known in the art (see e.g. Marcus-Sakura, Anal. Biochem. 1988, 172:289). Further, antisense molecules which bind directly to the DNA may be used. Antisense nucleic acids may be single or double stranded nucleic acids. Non-limiting examples of antisense nucleic acids include siRNAs (including their derivatives or pre-cursors, such as nucleotide analogs), short hairpin RNAs (shRNA), micro RNAs (miRNA), saRNAs (small activating RNAs) and small nucleolar RNAs (snoRNA) or certain of their derivatives or pre-cursors. Antisense nucleic acid molecules may stimulate RNA interference (RNAi).

Thus, an antisense nucleic acid cargo may interfere with transcription of target genes, interfere with translation of target mRNA and/or promote degradation of target mRNA. In some cases, an antisense nucleic acid is capable of inducing a reduction in expression of the target gene.

A “siRNA,” “small interfering RNA,” “small RNA,” or “RNAi” as provided herein, refers to a nucleic acid that forms a double stranded RNA, which double stranded RNA has the ability to reduce or inhibit expression of a gene or target gene when expressed in the same cell as the gene or target gene. The complementary portions of the nucleic acid that hybridize to form the double stranded molecule typically have substantial or complete identity. In one embodiment, a siRNA or RNAi is a nucleic acid that has substantial or complete identity to a target gene and forms a double stranded siRNA. In embodiments, the siRNA inhibits gene expression by interacting with a complementary cellular mRNA thereby interfering with the expression of the complementary mRNA. Typically, the nucleic acid is at least about 15-50 nucleotides in length (e.g., each complementary sequence of the double stranded siRNA is 15-50 nucleotides in length, and the double stranded siRNA is about 15-50 base pairs in length). In some embodiments, the length is 20-30 base nucleotides, preferably about 20-25 or about 24-29 nucleotides in length, e.g., 20, 21, 22, 23, 24, 25, 26, 27, 28, 29, or 30 nucleotides in length.

RNAi and siRNA are described in, for example, Dana et al., Int J Biomed Sci. 2017; 13(2): 48-57, herein incorporated by reference in its entirety. An antisense nucleic acid molecule may contain double-stranded RNA (dsRNA) or partially double-stranded RNA that is complementary to a target nucleic acid sequence, for example FHR-4. A double-stranded RNA molecule is formed by the complementary pairing between a first RNA portion and a second RNA portion within the molecule. The length of an RNA sequence (i.e. one portion) is generally less than 30 nucleotides in length (e.g. 29, 28, 27, 26, 25, 24, 23, 22, 21, 20, 19, 18, 17, 16, 15, 14, 13, 12, 11, 10 or fewer nucleotides). In some embodiments, the length of an RNA sequence is 18 to 24 nucleotides in length. In some siRNA molecules, the complementary first and second portions of the RNA molecule form the “stem” of a hairpin structure. The two portions can be joined by a linking sequence, which may form the “loop” in the hairpin structure. The linking sequence may vary in length and may be, for example, 5, 6, 7, 8, 9, 10, 11, 12, or 13 nucleotides in length. Suitable linking sequences are known in the art.

Suitable siRNA molecules for use in the methods of the present invention may be designed by schemes known in the art, see for example Elbashire et al., Nature, 2001 411:494-8; Amarzguioui et al., Biochem. Biophys. Res. Commun. 2004 316(4):1050-8; and Reynolds et al., Nat. Biotech. 2004, 22(3):326-30. Details for making siRNA molecules can be found in the websites of several commercial vendors such as Ambion, Dharmacon, GenScript, Invitrogen and OligoEngine. The sequence of any potential siRNA candidate generally can be checked for any possible matches to other nucleic acid sequences or polymorphisms of nucleic acid sequence using the BLAST alignment program (see the National Library of Medicine internet website). Typically, a number of siRNAs are generated and screened to obtain an effective drug candidate, see, U.S. Pat. No. 7,078,196. siRNAs can be expressed from a vector and/or produced chemically or synthetically. Synthetic RNAi can be obtained from commercial sources, for example, Invitrogen (Carlsbad, Calif.). RNAi vectors can also be obtained from commercial sources, for example, Invitrogen.

The nucleic acid molecule may be a miRNA. The term “miRNA” is used in accordance with its plain ordinary meaning and refers to a small non-coding RNA molecule capable of post-transcriptionally regulating gene expression. In one embodiment, a miRNA is a nucleic acid that has substantial or complete identity to a target gene. In some embodiments, the miRNA inhibits gene expression by interacting with a complementary cellular mRNA thereby interfering with the expression of the complementary mRNA. Typically, the miRNA is at least about 15-50 nucleotides in length (e.g., each complementary sequence of the miRNA is 15-50 nucleotides in length, and the miRNA is about 15-50 base pairs in length)In some cases, the nucleic acid is synthetic or recombinant.

The nucleic acid disclosed herein may comprise one or more modifications, or non-naturally occurring elements or nucleic acids. In preferred aspects, the nucleic acid comprises a 2′-O-methyl analog. In some cases, the nucleic acid includes a 3′ phosphorothioate internucleotide linkage or other locked nucleic acid (LNA). In some cases, the nucleic acid comprises an ARCA cap. Other chemically modified nucleic acids or nucleotides may be used, for example, 2′-position sugar modifications, 2′-O-methylation, 2′-Fluoro modifications, 2′NH2 modifications, 5-position pyrimidine modifications, 8-position purine modifications, modifications at exocyclic amines, substitution of 4-thiouridine, substitution of 5-bromo, or 5-iodo-uracil, backbone modifications, methylations, unusual base-pairing combinations such as isocytidine and isoguanidine and the like. Modifications can also include 3′ and 5′ modifications such as capping. For example, the nucleic acid may be PEGylated.

Nucleic acids useful in the methods of the invention include antisense oligonucleotides, mRNA, siRNAs or gRNAs that target oncogenic miRNAs (also known as oncomiRs) or transcription factors. The cargo may be a ribozyme or aptamer. In some cases, the nucleic acid is a plasmid.

The nucleic acid molecule may be an aptamer. The term “aptamer” as used herein refers to oligonucleotides (e.g. short oligonucleotides or deoxyribonucleotides), that bind (e.g. with high affinity and specificity) to proteins, peptides, and small molecules. Aptamers typically have defined secondary or tertiary structure owing to their propensity to form complementary base pairs and, thus, are often able to fold into diverse and intricate molecular structures. The three-dimensional structures are essential for aptamer binding affinity and specificity, and specific three-dimensional interactions drives the formation of aptamer-target complexes. Aptamers can be selected in vitro from very large libraries of randomized sequences by the process of systemic evolution of ligands by exponential enrichment (SELEX as described in Ellington AD, Szostak JW, Nature 1990, 346:818-822; Tuerk C, Gold L. Science 1990, 249:505-510) or by developing SOMAmers (slow off-rate modified aptamers) (Gold L et al. (2010) Aptamer-based multiplexed proteomic technology for biomarker discovery. PLoS ONE 5(12):e15004).

In certain aspects described herein, the cargo is an antisense oligonucleotide (ASO). The antisense oligonucleotide may be complementary to a miRNA or mRNA. The antisense oligonucleotide comprises at least a portion which is complementary in sequence to a target mRNA sequence. The antisense oligonucleotide may bind to, and thereby inhibit, the target sequence. For example, the antisense oligonucleotide may inhibit the translation process of the target sequence. The miRNA may be a miRNA associated with cancer (Oncomir). The miRNA may be miR-125b. The ASO may comprise or consist of the sequence 5′-UCACAAGUUAGGGUCUCAGGGA-3′.

In some aspects, the cargo is one or more components of a gene editing system. For example, a CRISPR/Cas9 gene editing system. For example, the cargo may include a nucleic acid which recognises a particular target sequence. The cargo may be a gRNA. Such gRNAs may be useful in CRISPR/Cas9 gene editing. The cargo may be a Cas9 mRNA or a plasmid encoding Cas9. Other gene editing molecules may be used as cargo, such as zinc finger nucleases (ZFNs) or Transcription activator-like effector nucleases (TALENs). The cargo may comprise a sequence engineered to target a particular nucleic acid sequence in a target cell. The gene editing molecule may specifically target a miRNA. For example, the gene editing molecule may be a gRNA that targets miR-125b. The gRNA may comprise or consist of the sequence 5′-CCUCACAAGUUAGGGUCUCA-3′.

In some embodiments the methods employ target gene editing using site-specific nucleases (SSNs). Gene editing using SSNs is reviewed e.g. in Eid and Mahfouz, Exp Mol Med. 2016 Oct; 48(10): e265, which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined, often with insertion or deletion of nucleotides. Alternatively DSBs may be repaired by highly homology-directed repair (HDR), in which a DNA template with ends homologous to the break site is supplied and introduced at the site of the DSB.

SSNs capable of being engineered to generate target nucleic acid sequence-specific DSBs include ZFNs, TALENs and clustered regularly interspaced palindromic repeats/CRISPR-associated-9 (CRISPR/Cas9) systems.

ZFN systems are reviewed e.g. in Umov et al., Nat Rev Genet. (2010) 11(9):636-46, which is hereby incorporated by reference in its entirety. ZFNs comprise a programmable Zinc Finger DNA-binding domain and a DNA-cleaving domain (e.g. a Fokl endonuclease domain). The DNA-binding domain may be identified by screening a Zince Finger array capable of binding to the target nucleic acid sequence. TALEN systems are reviewed e.g. in Mahfouz et al., Plant Biotechnol J. (2014) 12(8):1006-14, which is hereby incorporated by reference in its entirety. TALENs comprise a programmable DNA-binding TALE domain and a DNA-cleaving domain (e.g. a Fokl endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: “HD” binds to C, “NI” binds to A, “NG” binds to T and “NN” or “NK” binds to G (Moscou and Bogdanove, Science (2009) 326(5959):1501.).

CRISPR is an abbreviation of Clustered Regularly Interspaced Short Palindromic Repeats. The term was first used at a time when the origin and function of these sequences were not known and they were assumed to be prokaryotic in origin. CRISPR are segments of DNA containing short, repetitive base sequences in a palindromic repeat (the sequence of nucleotides is the same in both directions). Each repetition is followed by short segments of spacer DNA from previous integration of foreign DNA from a virus or plasmid. Small clusters of CAS (CRISPR-associated) genes are located next to CRISPR sequences. RNA harboring the spacer sequence helps Cas (CRISPR-associated) proteins recognize and cut foreign pathogenic DNA. Other RNA-guided Cas proteins cut foreign RNA. A simple version of the CRISPR/Cas system, CRISPR/Cas9, has been modified to edit genomes. By delivering the Cas9 nuclease and a synthetic guide RNA (gRNA) into a cell, the cell's genome can be cut at a desired location, allowing existing genes to be removed and/or new ones added. CRISPR-Cas systems fall into two classes. Class 1 systems use a complex of multiple Cas proteins to degrade foreign nucleic acids. Class 2 systems use a single large Cas protein for the same purpose. Class 1 is divided into types I, III, and IV; class 2 is divided into types II, V, and VI. CRISPR genome editing uses a type II CRISPR system.

In some aspects, the EV is loaded with a CRISPR related cargo. In other words, the EV is useful in a method involving gene editing, such as therapeutic gene editing. In some cases, the EV is useful for in vitro gene editing.

The cargo may be a guide RNA. The guide RNA may comprise a CRIPSR RNA (crRNA) and a trans-activating CRISPR RNA (tracrRNA). The crRNA contains a guide RNA that locates the correct section of host DNA along with a region that binds to tracrRNA forming an active complex. The tracrRNA binds to crRNA and forms the active complex. The gRNA combines both the tracrRNA and a crRNA, thereby encoding an active complex. The gRNA may comprise multiple crRNAs and tracrRNAs. The gRNA may be designed to bind to a sequence or gene of interest. The gRNA may target a gene for cleavage. Optionally, an optional section of DNA repair template is included. The repair template may be utilized in either non-homologous end joining (NHEJ) or homology directed repair (HDR).

The cargo may be a nuclease, such as a Cas9 nuclease. The nuclease is a protein whose active form is able to modify DNA. Nuclease variants are capable of single strand nicking, double strand break, DNA binding or other different functions. The nuclease recognises a DNA site, allowing for site specific DNA editing.

The gRNA and nuclease may be encoded on a plasmid. In other words, the EV cargo may comprise a plasmid that encodes both the gRNA and the nuclease. In some cases, an EV contains the gRNA and another EV contains or encodes the nuclease. In some cases, an EV contains a plasmid encoding the gRNA, and a plasmid encoding the nuclease. Thus, in some aspects, a composition is provided comprising EVs, wherein a portion of the EVs comprise or encode the nuclease such as Cas9, and a portion of the EVs comprise or encode the gRNA. In some cases, a composition containing EVs that comprise or encode the gRNA and a composition containing EVs that encode or contain the nuclease are co-administered. In some cases, the composition comprises EVS wherein the EVs contain an oligonucleotide that encodes both a gRNA and a nuclease.

CRISPR/Cas9 and related systems e.g. CRISPR/Cpf1, CRISPR/C2c1, CRISPR/C2c2 and CRISPR/C2c3 are reviewed e.g. in Nakade et al., Bioengineered (2017) 8(3):265-273, which is hereby incorporated by reference in its entirety. These systems comprise an endonuclease (e.g. Cas9, Cpf1 etc.) and the single-guide RNA (sgRNA) molecule. The sgRNA can be engineered to target endonuclease activity to nucleic acid sequences of interest.

In some cases, the nucleic acid encodes or targets one or more dedifferentiation factors, such as one or more nucleic acids encoding the “Yamanaka factors”, Oct4, Sox2, Klf4 and Myc.

In some cases, the cargo is a peptide or protein. It may be a recombinant peptide or protein. Suitable peptides or proteins include enzymes, such as gene editing enzymes such as Cas9, a ZFN, or a TALEN.

Suitable small molecules include cytotoxic reagents and kinase inhibitors. The small molecule may comprise a fluorescent probe and/or a metal. For example, the cargo may comprise a superparamagnetic particle such as an iron oxide particle. The cargo may be an ultra-small superparamagnetic iron oxide particle such as an iron oxide nanoparticle.

In some cases, the cargo is a detectable moiety such as a fluorescent dextran. The cargo may be radioactively labelled.

Cargo may be loaded into the extracellular vesicles by electroporation. Electroporation, or electropermeabilization, is a microbiology technique in which an electrical field is applied to cells in order to increase the permeability of the cell membrane, allowing chemicals, drugs, or DNA to be introduced into the cell. In other words, the extracellular vesicles may be induced or force to encapsulate the cargo by electroporation. As such, methods disclosed herein may involve a step of electroporating an extracellular vesicle in the presence of a cargo molecule, or electroporating a mixture of extracellular vesicles and cargo molecules.

In other methods disclosed herein, cargo is loaded into the extracellular vesicles by sonication, ultrasound, lipofection or hypotonic dialysis.

The cargo may be loaded into the extracellular vesicle before or after the extracellular vesicle has been tagged.

Compositions

Disclosed herein are compositions comprising extracellular vesicles.

The compositions may comprise between 10⁶ to 10¹⁴ particles per ml. The compositions may comprise at least 10⁵ particles per ml, at least 10⁶ particles per ml, at least at least 10⁷ particles per ml, at least 10⁸ particles per ml, at least 10⁹ particles per ml, at least 10¹⁰ particles per ml, at least 10¹¹ particles per ml, at least 10¹² particles per ml, at least 10¹³ particles per ml or at least 10¹⁴ particles per ml.

The composition may comprise extracellular vesicles have substantially homologous dimensions. For example, the extracellular vesicles may have diameters ranging from 100-500 nm. In some cases, a composition of microvesicles comprises microvesicles with diameters ranging from 50-1000 nm, from 101-1000 nm, from 101-750 nm, from 101-500 nm, or from 100-300 nm, or from 101-300 nm. Preferably, the diameters are from 100-300 nm. In some compositions, the mean diameter of the microvesicles is 100-300 nm, preferably 150-250 nm, preferably about 200 nm.

Although it is desirable for the tag to be linked to substantially all of the extracellular vesicles in a composition, compositions disclosed herein may comprise extracellular vesicles in which at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, or at least 97% of the extracellular vesicles comprise the tag. Preferably, at least 85%, at least 90%, at least 95%, at least 96% or at least 97% of the extracellular vesicles comprise the tag. In some cases, different extracellular vesicles within the composition comprise different tags. In some cases, the extracellular vesicles comprise the same, or substantially the same, tag.

In some compositions, in addition to comprising tag, the extracellular vesicles contain a cargo. Although it is desirable in such compositions for the cargo to be encapsulated into substantially all of the extracellular vesicles in a composition, compositions disclosed herein may comprise extracellular vesicles in which at least 30%, at least 35%, at least 40%, at least 45%, at least 50%, at least 55%, at least 60%, at least 65%, at least 70%, at least 75%, at least 80%, at least 85%, at least 90%, at least 95%, or at least 97% of the extracellular vesicles contain the cargo. Preferably, at least 85%, at least 90%, at least 95%, or at least 97% of the extracellular vesicles contain the cargo. In some cases, different extracellular vesicles within the composition contain different cargo. In some cases, the extracellular vesicles contain the same, or substantially the same, cargo molecule.

The composition may be a pharmaceutical composition. The composition may comprise one or more extracellular vesicle, and optionally a pharmaceutically acceptable carrier. Pharmaceutical compositions may be formulated for administration by a particular route of administration. For example, the pharmaceutical composition may be formulated for intravenous, intratumoral, intraperitoneal, intradermal, subcutaneous, intranasal or other administration route.

Compositions may comprise a buffer solution. Compositions may comprise a preservative compound. Compositions may comprise a pharmaceutically acceptable carrier.

Methods of Treatment and Uses of Extracellular vesicles

Extracellular vesicles disclosed herein are useful in methods of treatment. In particular, the methods are useful for treating a subject suffering from a disorder associated with a target gene, the method comprising the step of administering an effective amount of a modified extracellular vesicle to said subject, wherein the modified extracellular vesicle comprises a binding molecule on its surface and encapsulates a non-endogenous substance for interacting with the target gene in a target cell. The non-endogenous substance may be a nucleic acid for said treatment.

The extracellular vesicles disclosed herein are particularly useful for the treatment of a genetic disorder, inflammatory disease, cancer, autoimmune disorder, cardiovascular disease or a gastrointestinal disease. In some cases, the disorder is a genetic disorder selected from thalassemia, sickle cell anemia, or genetic metabolic disorder. In some cases, the extracellular vesicles are useful for treating a disorder of the liver, bone marrow, lung, spleen, brain, pancreas, stomach or intestine.

In certain aspects, the extracellular vesicles are useful for the treatment of cancer. Extracellular vesicles disclosed herein may be useful for inhibiting the growth or proliferation of cancerous cells. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine.

The target cell depends on the disorder to be treated. For example, the target cell may be a breast cancer cell, a colorectal cancer cell, a lung cancer cell, a kidney cancer cell or the like. The cargo may be a nucleic acid for inhibiting or enhancing the expression of the target gene, or performing gene editing to silence the particular gene.

Extracellular vesicles and compositions described herein may be administered, or formulated for administration, by a number of routes, including but not limited to systemic, intratumoral, intraperitoneal, parenteral, intravenous, intra-arterial, intradermal, subcutaneous, intramuscular, oral and nasal. Preferably, the extracellular vesicles are administered by a route selected from intratumoral, intraperitoneal or intravenous. The medicaments and compositions may be formulated in fluid or solid form. Fluid formulations may be formulated for administration by injection to a selected region of the human or animal body.

The extracellular vesicle may comprise a tag that binds to a molecule on the surface of the cell or tissue to be treated. The tag may specifically bind to the cell or tissue to be treated. The extracellular vesicle may comprise a therapeutic cargo. The therapeutic cargo may be a non-endogenous substance for interacting with a target gene in a target cell.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Extracellular vesicles may be administered alone or in combination with other treatments, either simultaneously or sequentially dependent upon the condition to be treated.

Extracellular vesicles loaded with a cargo as described herein may be used to deliver that cargo to a target cell. In some cases, the method is an in vitro method. In particularly preferred in vitro methods the cargo is a labelling molecule or a plasmid.

The subject to be treated may be any animal or human. The subject is preferably mammalian, more preferably human. The subject may be a non-human mammal, but is more preferably human. The subject may be male or female. The subject may be a patient. Therapeutic uses may be in humans or animals (veterinary use).

Kit

Also disclosed herein are kits comprising extracellular vesicles, or for use in tagging extracellular vesicles. The kit may comprise one or more components selected from one or more extracellular vesicles, a tag or nucleic acid encoding the tag such as an expression vector for expressing the tag in a cell culture, a cargo or non-endogenous molecule for encapsulation in the extracellular vesicle, a protein ligase and optionally a protein ligase buffer.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1

For therapeutic delivery, many research groups have attempted to produce EVs from cancer cell lines and stem cells which are very costly due to the large-scale cell culture. Moreover, EVs from cancer and stem cells may contain oncogenic proteins or growth factors that promote cancer growth. EVs from plasma and blood cells are safer for cancer therapies. We have recently developed a robust method for large scale purification of EVs from red blood cells (RBCs) and incorporation of RNAs in these EVs for gene therapies against cancer including acute myeloid leukemia (AML) and triple negative breast cancer (TNBC). We have shown that RBCEVs are taken up very well by both AML and TNBC cells and confer better transfection efficiency with lower toxicity than commercial transfection reagents. We also observed the uptake of RBCEVs in vivo where RBCEVs deliver antisense oligonucleotides (ASOs) that inhibits oncogenic miR-125b and suppressed the progression of AML and TNBCs. RBCEVs are also used to deliver Cas9 mRNA and gRNA for genome editing in leukemia cells. This platform is very promising for gene therapies against cancer.

To make EV-based therapy more specific, EVs are often engineered to have peptides or antibodies that bind specifically to certain target cells.⁵ Usually, these peptides or antibodies are expressed in donor cells from plasmids that are transfected or transduced using retrovirus or lentivirus followed by an antibiotic-based or fluorescence-based selection.³ These methods post a high risk of horizontal gene transfer as the highly expressed plasmids are likely incorporated into EVs and eventually transferred to the target cells. Genetic elements in the plasmids may cause oncogenesis. If stable cell lines are made to produce EVs, abundant oncogenic factors including mutant DNAs, RNAs and proteins are packed in EVs and deliver to the target cells the risk of tumorigenesis. On the other hand, genetic engineering methods are not applicable to RBCs as plasmids cannot be transcribed in RBCs because of the lack of ribosome. It is also not applicable to stem cells and primary cells that are hard to transfect or transduce.

Recently, there is a new method for coating EVs with antibodies fused to a C1C2 domain of lactadherin that bind to phosphatidylserine (PS) on the surface of EVs.⁶ This method allows conjugation of EVs with antibodies without any genetic modification.⁶ However, C1C2 is a hydrophobic protein hence requires a tedious purification method in mammalian cells and storage in bovine serum albumin containing buffer. Moreover, the conjugation of EVs with C1C2-fusion antibodies is based on the affinity binding between C1C2 and PS that is transient. To have a stable and permanent conjugation of EVs with targeting or therapeutic proteins, we need conjugation methods that generate covalent bonds using chemical or enzymatic reactions.

We previously used the transpeptidase sortase to covalently attach peptides and single domain antibodies (sdAbs), proteins with single immunoglobulin domain derived from humans, camels or cartilaginous fishes, to the surface of RBCs that are engineered with a sortase-recognition motif in RBC surface proteins.⁷ A more recent study revealed that sortase can link many peptides, all extended at their C-termini by a sortase recognition sequence, to unmodified proteins with N-terminal glycine residues on the RBC surface.8 Hence, sortase can covalently ligate native proteins with N-terminal glycine on cell surface with proteins or peptides carrying C-terminal sortase tag.⁸ We hypothesize that native proteins with N-terminal glycine and /or side chain amino group on the surface of RBCEV may act as the substrate for sortase. Therefore, we can use sortase and similar protein ligase enzymes to coat RBCEVs with peptides, small molecules, proteins and sdAbs.

Here we describe a method for enzymatic modification of EV surface using sortase A. Peptides conjugated with small molecules (such as biotin) and sdAbs containing sortase-binding sites are attached to proteins on EV surface via stable covalent bonds. This enables targeted delivery of EVs to specific cell types for therapeutic purposes.

RESULTS

Conjugation of EVs with sdAbs using Sortase A

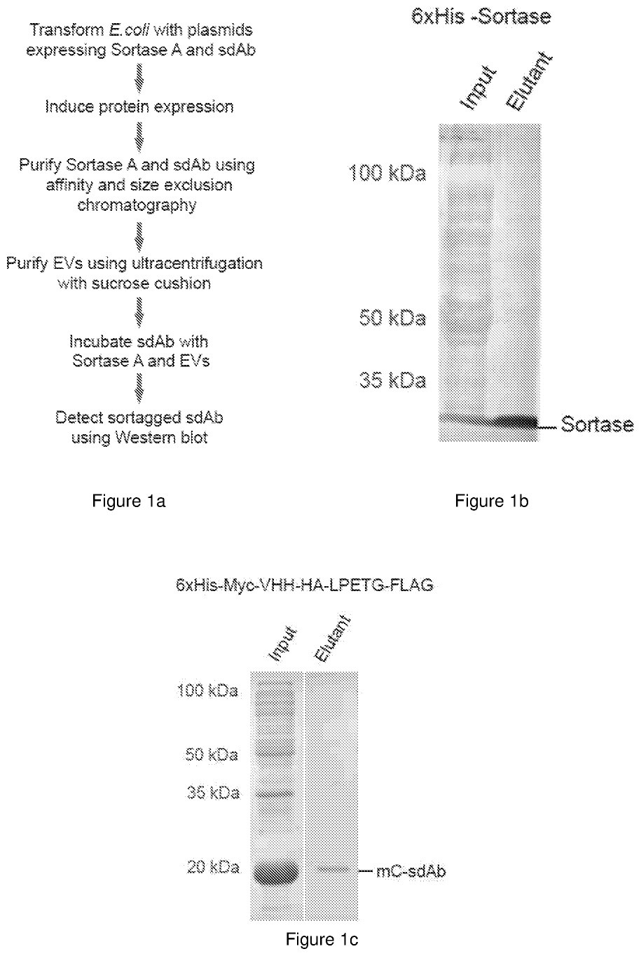

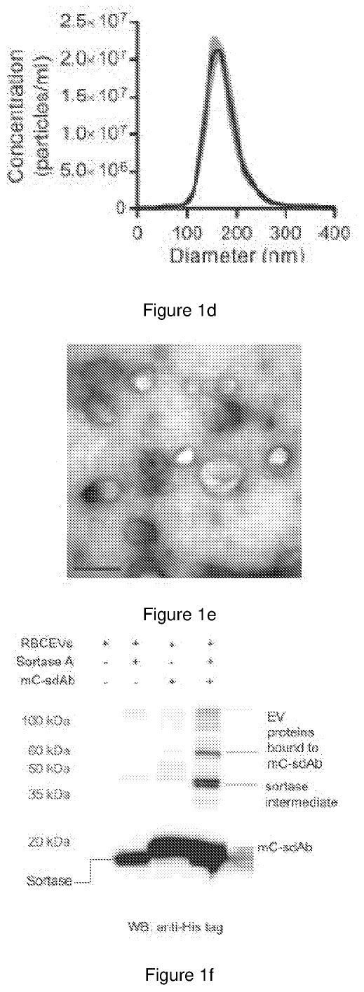

The inventors developed a simple workflow for conjugation of EVs with peptides and sdAbs, including the purification of each component, sortagging reactions and detection of sortagged proteins on EVs (FIG. 1A). Sortase A was expressed with His tags in E.coli and purified using affinity and size exclusion chromatography to ˜27 mg protein from 1 L bacteria culture with ˜100% purity (FIG. 1B). Similarly, a sdAb specific to mCherry (mC-sdAb) was expressed with His tags, FLAG tag, HA tag and a sortase binding site

(LPETG) in E.coli and purified using the same protocol as for sortase A with a yield of 8 mg pure protein from 1 L culture. Fifteen or more amino acids are inserted between VHH and the sortase binding site to increase the accessibility and flexibility of the sortase binding site to sortase. The sdAb appeared as a clear 20 kDa single band after purification (FIG. 1C).

RBCEVs were purified according to our established protocol including stimulation of EV release using calcium ionophore, differential centrifugation to remove the cells and debris and three times ultracentrifugation including once with sucrose cushion.⁴ Nanosight analysis demonstrated that the RBCEVs purified from multiple donors were very consistent with the diameter range from 100 to 300 nm (FIG. 1 D). The EVs appeared as clear double-layer membrane vesicles, with the typical cup shape, and no protein aggregation under a transmission electron microscope (FIG. 1E).

Using an anti-His tag antibody (which also binds to VHH) for Western blot analysis, we found sortase A and mC-sdAb as 18 kDa and 20 kDa bands, respectively (FIG. 1F). Remarkably, after incubating RBCEVs with sortase A and mC-sdAb, we observed additional bands at ˜40, and 70 kDa that were absent after the incubation of RBCEVs with only sortase A or only mC-sdAb. The new bands appeared after the sortagging reaction indicated the association of mC-sdAb with sortase A and proteins in RBCEVs. This association was stable in denaturing condition, suggesting that mC-sdAb bound to the proteins on RBCEVs through covalent bonds generated by the sortagging reaction.

Conjugation of EVs with Peptides using Sortase A

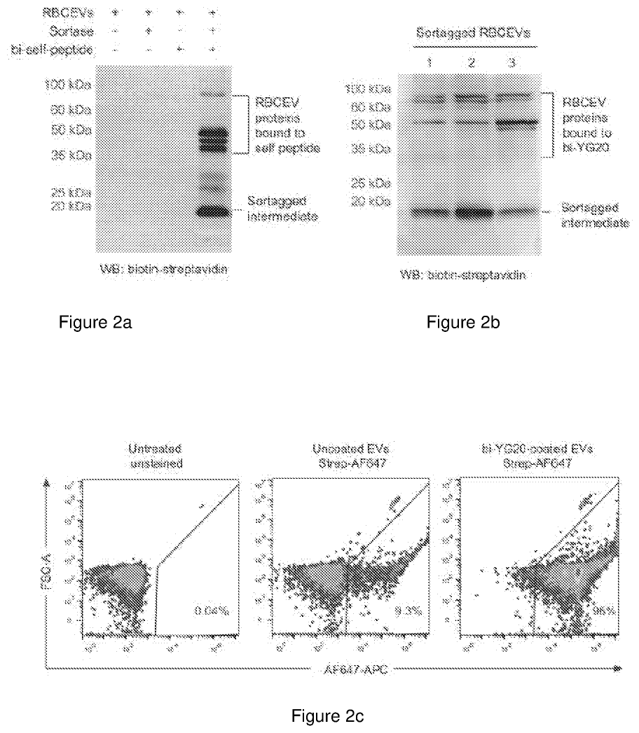

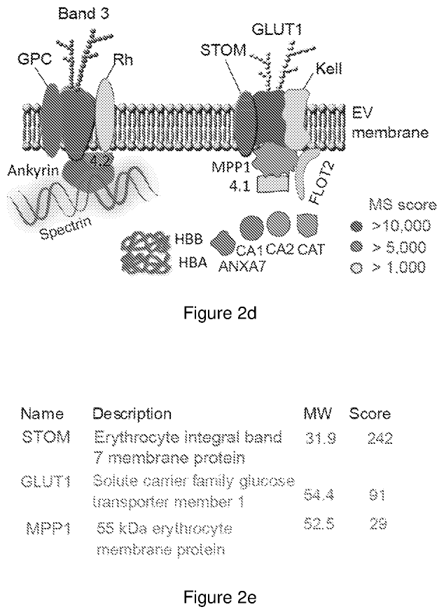

We further tested if RBCEVs can be sortagged with peptides. We added a sortase A binding site to the C terminus of a peptide that is known as “self peptide” because it was derived from CD47 which is the “don't eat me” signal to avoid phagocytosis by macrophages.¹⁰ This peptide also has a biotin tag at the N terminus. In a Western blot analysis using HRP-conjugated streptavidin, we could not detect the peptide when it was loaded alone due to its small size (2.4 kDa) but we observed a thick band at ˜20 kDa corresponding to the size of the sortase plus the peptide when we incubated RBCEVs with the peptide and sortase A hence this should be the intermediate product of the sortagging reaction (FIG. 2A). In addition, we observed multiple bands ranging from 25 kDa to 75 kDa in RBCEVs incubated with the peptides and sortase A (FIG. 2A). These bands should be proteins on the surface of RBCEVs that were sortagged with the biotinylated peptides. Similarly, we added the sortase binding site to another peptide well-known for its binding to epidermal growth factor receptor (EGFR), a surface protein highly expressed in many types of solid cancer.¹¹ A biotin tag was also added to the N terminus of the peptide (hereafter called bi-YG20 peptide) through chemical synthesis. We sortagged the YG20 peptide to 3 different batches of RBCEVs purified independently from 3 donors and found similar bands of sortagged proteins in the 3 samples, except one additional band in the third sample. This observation suggested that some abundant proteins on the surface RBCEVs from every donor contain N-terminal Glycine residues that consistently reacted with sortase A.

To estimate the efficiency of sortagging, we incubated the bi-YG20-coated EVs with latex beads and stained the beads with Alexa Fluor 647 (AF647) conjugated streptavidin. FACS analysis demonstrated that 96% of the beads was positive for AF647, indicating that most of the EVs were successfully conjugated with the biotinylated peptide (FIG. 2C).

Using mass spectrometry, we identified nearly 20 proteins that were highly abundant in RBCEVs including 12 membrane proteins that are also known for their expression in RBCs (FIG. 2D). To identify membrane proteins that reacted to sortase A, we used streptavidin beads to pull down proteins in the RBCEV membrane lysate that were conjugated with the biotinylated peptide. We identified 3 proteins including STOM, GLUT1 and MPP1 that were enriched in the biotin-streptavidin complex (FIG. 2E). These proteins have similar molecular weights (31.9, 54.4 and 52.5 kDa) as some of the proteins observed using Western blot, and they are among the abundant proteins detected in RBCEVs, hence they are likely among the proteins that reacted to sortase A.

Sortagging EVs with an EGFR-Binding Peptide Promotes the Uptake of the EVs by EGFR-Positive Breast Cancer Cells

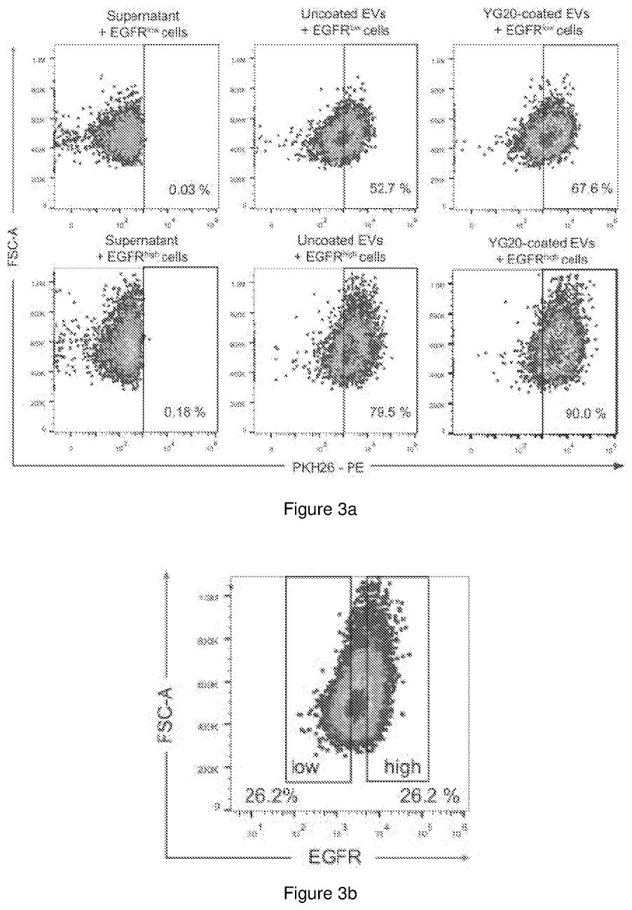

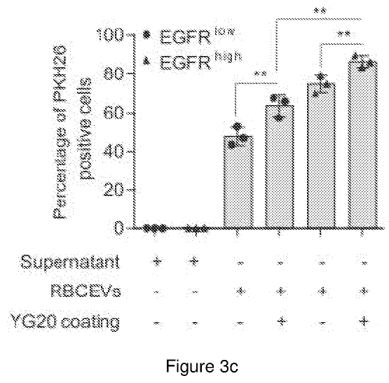

To test the uptake of EVs by breast cancer cells, we labeled RBCEVs with PKH26, a fluorescent membrane dye and sortagged the labeled RBCEVs with bi-YG20 peptide as described above. The labeled and sortagged RBCEVs were washed extensively with 2 rounds of ultracentrifugation including once with a sucrose cushion. We incubated SKBR3 cells with a suboptimal dose of the labeled RBCEVs (half of what we used for MOLM13 cells that showed 99% uptake)⁴ and analyzed PHK26 fluorescence in the cells after 24 hours of incubation (FIG. 3A). The fluorescent background was determined based on the supernatant of the last wash of the labeled RBCEVs. To test if the expression of EGFR is important for the uptake, we also stained the cells with an anti-EGFR antibody conjugated with FITC and gated two population of SKBR3 cells: one with low EGFR expression and one with high EGFR expression (FIG. 3B). As the result, the percentage of PKH26 positive cells was significantly higher in the SKBR3 cells treated with the bi-YG20-coated RBCEVs compared to the treatment with uncoated RBCEVs in both EGFR^{low} and EGFR^{high} populations (FIG. 3A, 3C). Higher expression of EGFR also made a significant difference in the uptake of the bi-YG20-coated RBCEVs (FIG. 3A, 3C). Hence, conjugation of RBCEVs with YG20 peptide promoted specific uptake of the EVs by EGFR positive breast cancer cells.

Conjugation of RBCEVs with Peptides using OaAEP1 Ligase

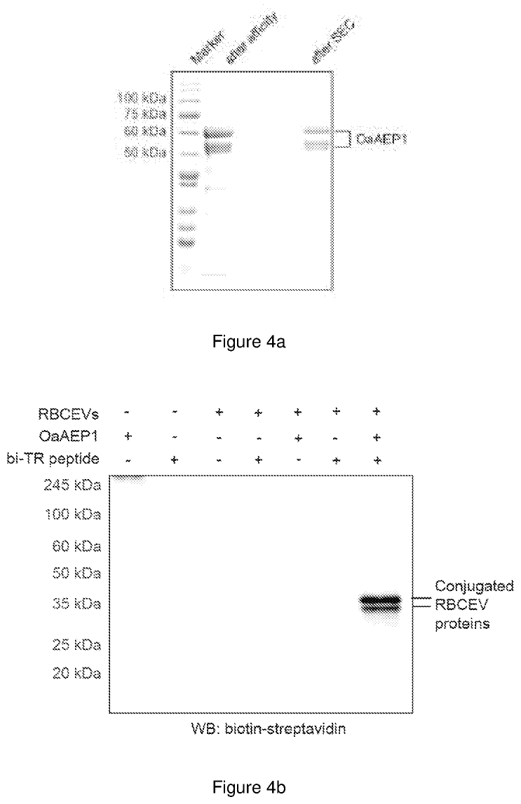

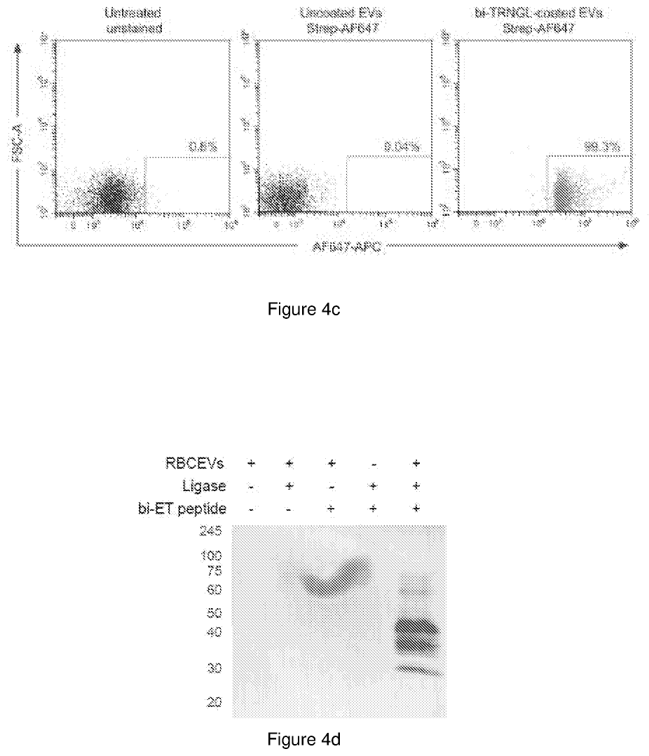

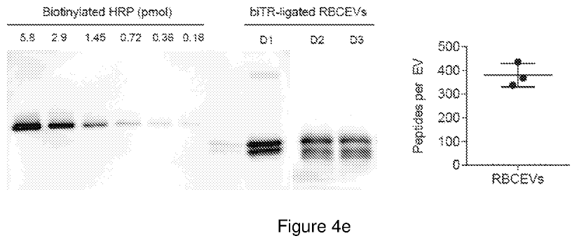

We further tested OaAEP1, a protein ligase, for conjugation of RBCEVs with peptides bearing TRNGL sequence. Here we used a variant of OaAEP1, with a Cys247Ala modification, that has a fast catalytic kinetics.¹² We purified OaAEP1 using affinity chromatography and SEC and obtained a pure enzyme with and without the His-Ub tag (FIG. 4A). The enzyme was incubated with RBCEVs and/or a peptide containing the OaAEP1 binding sequence “NGL”. The reaction of RBCEVs with the peptide led to multiple protein bands ranging from 35 kDa to ˜55 kDa to 200 kDa, detected with HRP-conjugated streptavidin, even after ligated RBCEVs were subjected to 3 extensive washes (FIG. 4B). These bands are different from the bands appeared from the sortagging reaction probably because OaAEP1 ligase only acts on proteins that have both glycine and leucine (GL) at the C terminus. Using FACS analysis, we found that the efficiency of RBCEV conjugation was 99.3% as the percentage of RBCEVs appeared to have biotin after the ligation reaction with bi-TRNGL peptide (FIG. 4C). We further tested the ligation of RBCEVs with a biotinylated EGFR-targeting peptide containing a ligase-binding site (NGL). Our Western blot analysis revealed prominent bands of 30-45 kDa after the ligation and 3 washes (FIG. 4D).

To quantify the number of peptides ligated on RBCEVs, we compared the intensity of biotin signals from the ligated RBCEV proteins to a serial dilution of biotinylated HRP. This comparison indicated that there were ˜380 copies of TR peptide ligated to each RBCEV, as an average of RBCEVs from 3 different blood donors (FIG. 4E).

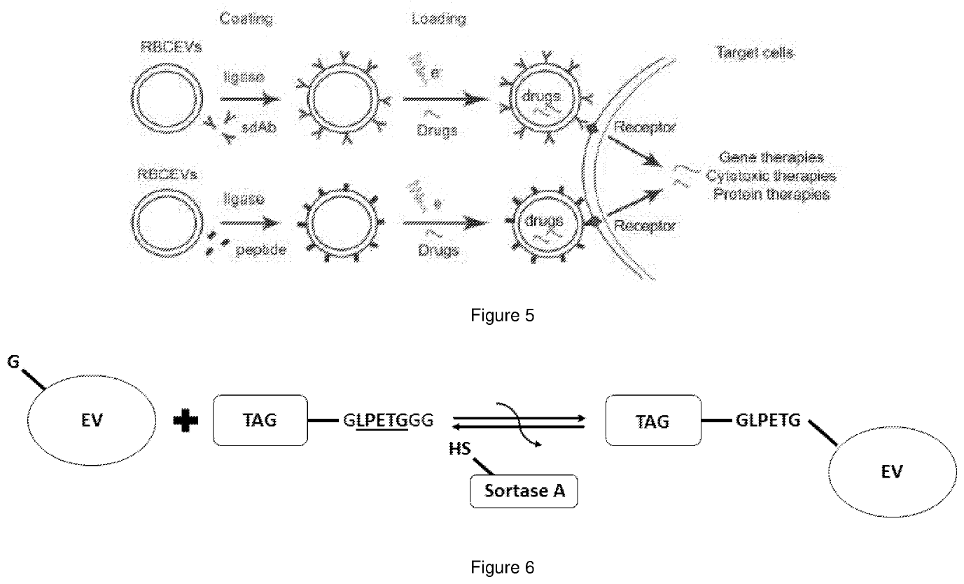

These data demonstrated a new approach for conjugation of EVs with sdAbs and peptides as the tags that mediate specific uptake of the EVs by the targeted cell types such as tumor cells for cancer treatments. This approach may facilitate the specific delivery of therapeutic molecules such as RNAs and DNAs for gene therapies, proteins for enzyme replacement therapies or vaccination, small cytotoxic molecules for cancer treatments, etc with reduced side effects (FIG. 5-6). In addition, the peptides and antibodies coated on the surface of EVs can also be applied directly to diagnosis and therapies.

Ligation of Leukemia EVs with Peptides using OaAEP1 Ligase

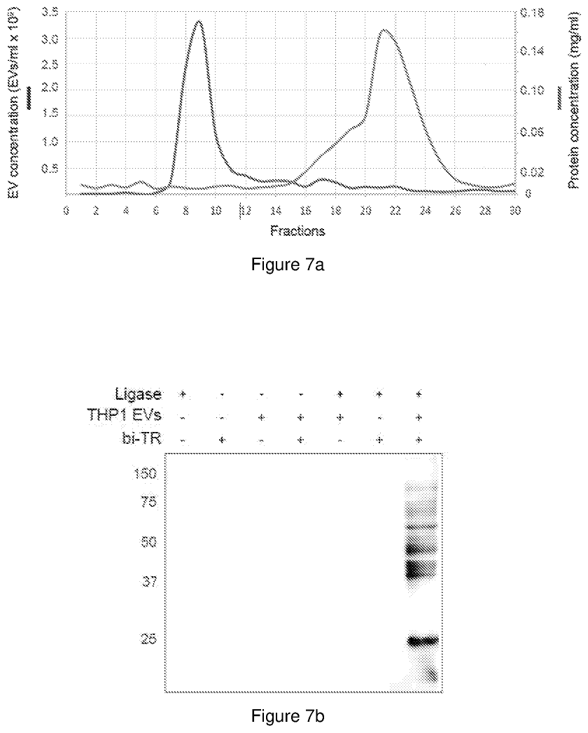

To validate the application of OaAEP1 ligase to modify other types of EVs, we isolated EVs from leukemia THP1 cells. THP1 cells were cultured with medium containing 10% EV-free FBS and treated with calcium ionophore overnight and the culture supernatant was centrifuged multiple times at increasing speeds to remove cells and debris. THP1 EVs were isolated using ultracentrifugation with sucrose cushion then further passed through an SEC column for a complete removal of serum proteins (FIG. 7A). Using the same ligation protocol optimized for RBCEVs, we ligated THP1 EVs to biotinylated TRNGL peptide, resulting in multiple ligated protein bands from 25 to 75 kDa (FIG. 7B). This pattern is different from the ligated protein bands on RBCEVs as THP1 EVs may display different proteins with N-terminal GL on their membrane.

Sortagging EVs with an EGFR-Binding Peptide Promotes the Uptake of the EVs by EGFR-Positive Lung Cancer Cells

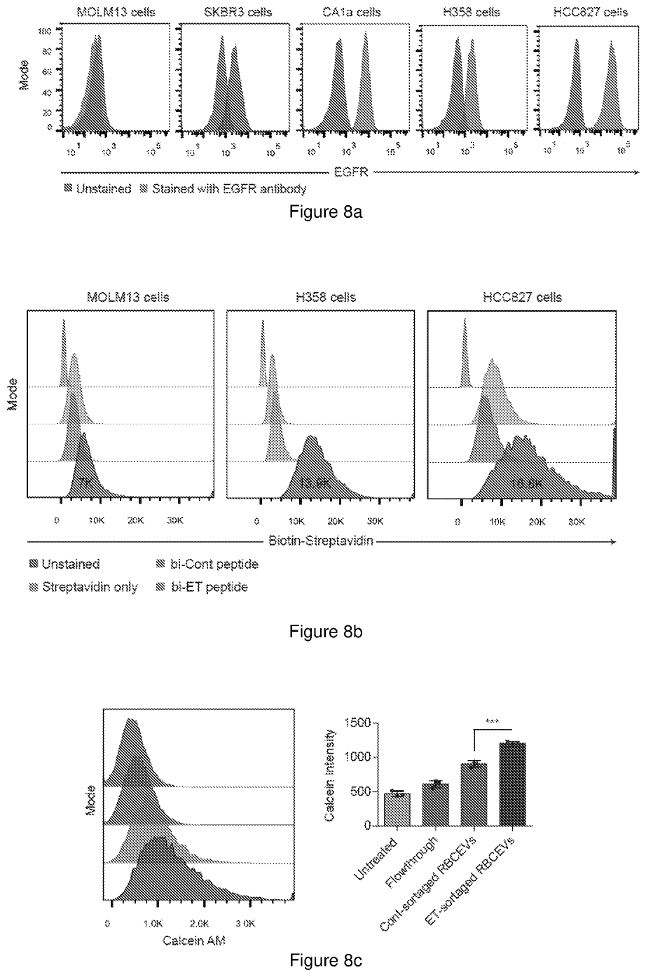

We further examined the expression of EGFR in 5 different human cell lines and found that EGFR was negative in MOLM13 and abundant in the solid cancer cells including breast cancer SKBR3 and CA1a cells, lung cancer H358 and HCC827 cells (FIG. 8A). Using FACS analysis of biotin-streptavidin, we found that biotinylated EGFR-targeting peptide bound to the surface of lung cancer H358 and HCC827 cells but not MOLM13 cells, relative to a streptavidin only control (FIG. 8B). A biotinylated control peptide with scrambled sequence did not bind to any of the tested cell lines.

To test the uptake of RBCEVs by the cells, we labelled RBCEVs with Calcein AM, a fluorescent dye, and sortagged the labelled RBCEVs with biotinylated EGFR-targeting peptide as described above. The labelled and sortagged RBCEVs were washed extensively with SEC and 2 rounds of centrifugation. We incubated H358 cells with a suboptimal dose of the labelled RBCEVs and analysed Calcein AM fluorescence in the cells after 2 hours of incubation (FIG. 8C). The fluorescent background was determined based on the supernatant of the last wash (flowthrough) of the labelled RBCEVs. As the result, the percentage of Calcein AM positive cells was significantly higher in the H358 cells treated with the EGFR-targeting peptide-coated RBCEVs compared to the treatment with control-peptide-coated RBCEVs (FIG. 8C). Hence, conjugation of RBCEVs with EGFR-targeting peptide promoted specific uptake of the EVs by EGFR positive lung cancer cells.

Ligase-Mediated Conjugation of RBCEVs with EGFR-Targeting Peptides Enhances the Specific Uptake of RBCEVs through Clathrin-Mediated Endocytosis

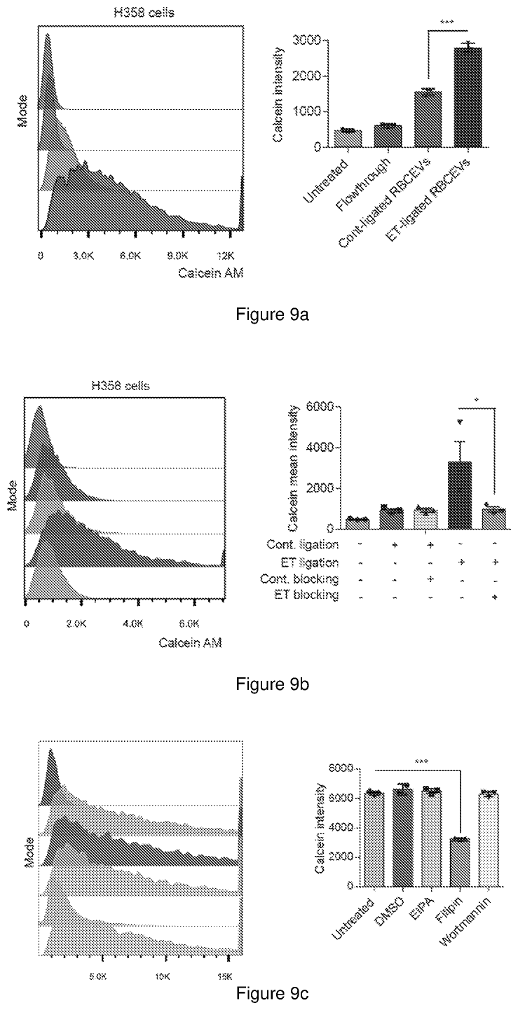

We repeated the above experiment using OaEAP1 ligase instead of sortase A. As expected, the uptake of RBCEVs by H358 cells significantly increased with the ligation of EGFR-targeting peptide compared to the control peptide (FIG. 9A). To examine the specificity of the uptake, we added a high concentration of free EGFR-targeting peptide to the incubation of H358 cells with EGFR-targeting peptide-ligated RBCEVs. The free peptide competed for binding to EGFR hence blocking the effect of the ligated EGFR-targeting peptide on RBCEVs (FIG. 9B), suggesting that the increase in EGFR-targeting peptide-ligated RBCEVs required EGFR binding.

To identify the route of RBCEV uptake, we added 3 different endocytosis inhibitors to the incubation of H358 cells with EGFR-targeting peptide-ligated RBCEVs. As the result, only Filipin, which blocks clathrin-mediated endocytosis, could reduce the uptake of ET-ligated RBCEVs (FIG. 9C). Therefore, the uptake of EGFR-targeting peptide-ligated RBCEVs was mediated by clathrin-mediated endocytosis.

Conjugation of RBCEVs with EGFR-Targeting Peptides Lead to an Enrichment of RBCEVs in EGFR-Positive Lung Tumours

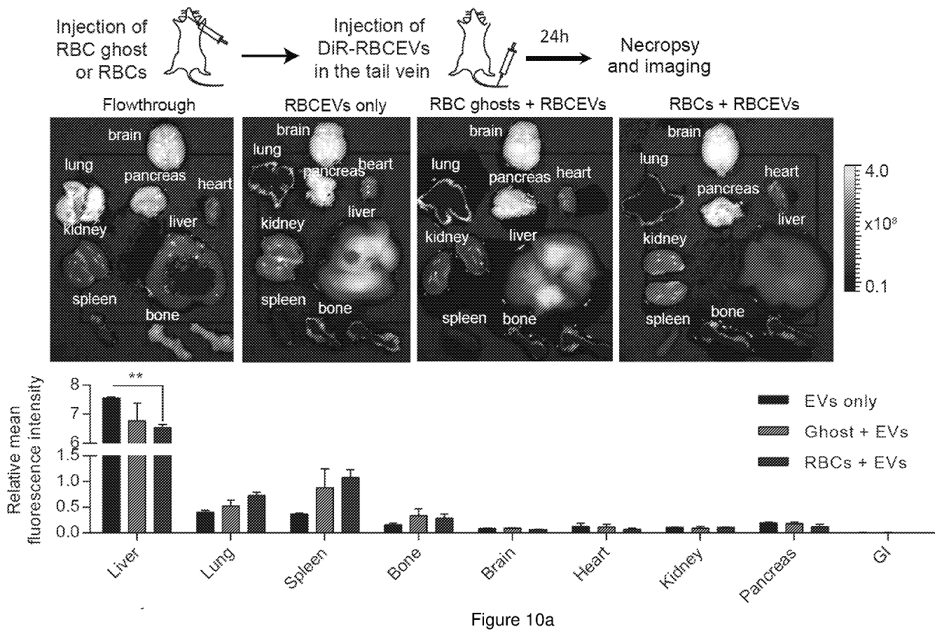

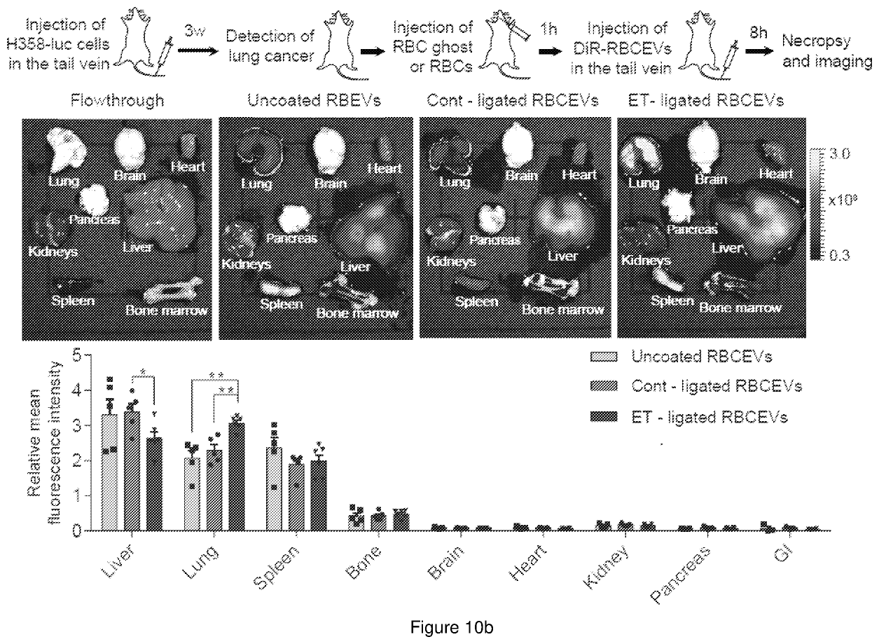

As RBCEVs usually accumulate in the liver due to the uptake by Kupffer cells, we sought to prevent rapid clearance of RBCEVs by preconditioning the mice with a dose of human RBCs or RBC ghosts (membrane of RBCs) before the injection of RBCEVs (FIG. 10A). We observed that RBCs were better than RBC ghosts in reducing the uptake of RBCEVs in the liver and increasing the uptake of RBCEVs in the lung and spleen. To generate an in vivo model of lung cancer, we injected luciferase-labelled H358 cells into the tail vein of NSG mice (FIG. 10B). After 3 weeks, when tumour cells were detected in the lung, we treated the mice with DiR-labelled RBCEVs and observed the biodistribution of the EVs using fluorescent imaging. Bioluminescence of tumour cells were detected consistently in the lung of NSG mice 3 weeks after the injection of H358-luciferase cells but no signal was detected in other organs except occasionally the tails due to residual cells from the tail vein injection. RBCEVs were conjugated with a control peptide or EGFR-targeting peptide then labelled with DiR fluorescent dye and washed extensively using SEC and centrifugation. Uncoated or coated RBCEVs were quantified using a haemoglobin assay and injected equally in the tail vein of preconditioned mice. The flowthrough of the EV wash was used to determine the fluorescent background. Eight hours after RBCEV injections, we observed distribution of uncoated RBCEVs to the spleen, liver, lung and bone (FIG. 10B). Peptide-coated RBCEVs showed uptake in the same organs. However, the accumulation of EGFR-targeting peptide-ligated RBCEVs significantly increased in the lung and reduced in the liver compared to the control-ligated RBCEVs (FIG. 10B). These data suggest that EGFR-targeting peptide drove RBCEVs to lung tumours expressing EGFR.

Conjugation with a Self-Peptide Prevents Phagocytosis of RBCEVs and Enhances the Availability of RBCEVs in the Circulation.

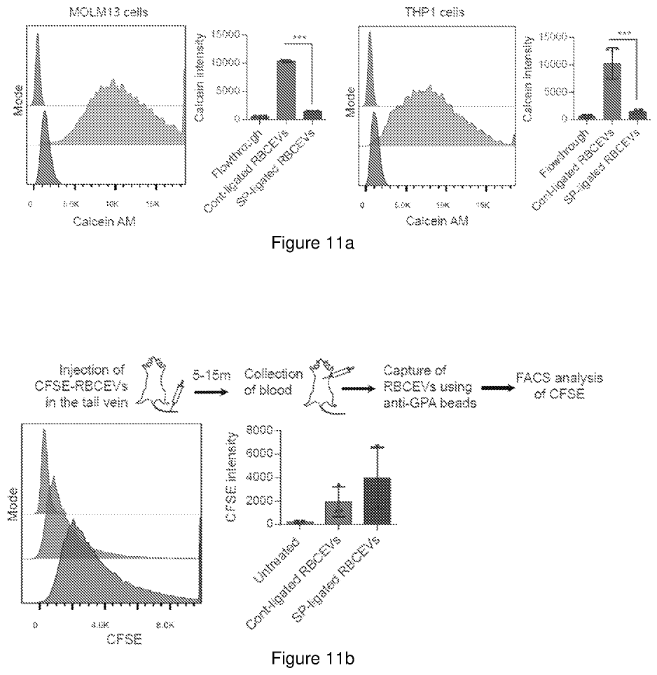

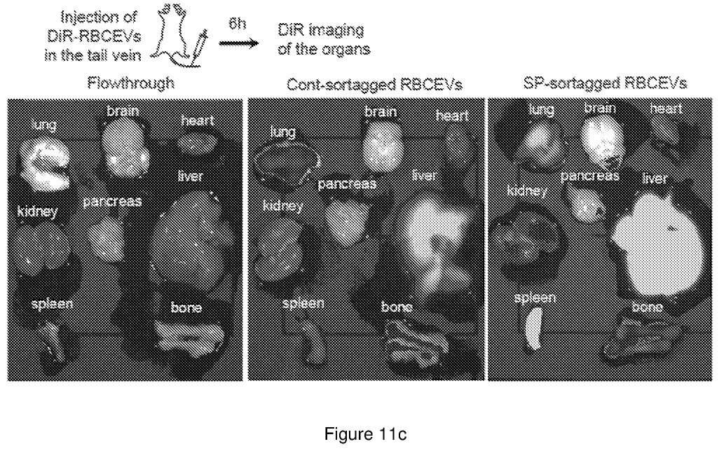

Similar to FIG. 2A, we conjugated RBCEVs with the self peptide but using OaAEP1 ligase instead of sortase A. Interestingly, ligation with the self peptide significantly reduced the uptake of RBCEVs by monocytes MOLM13 and THP1 cells (FIG. 11A-B).

We further labelled self-peptide-coated RBCEVs with CFSE and injected them in the tail vein of NSG mice. After 5 minutes, we captured RBCEVs in the blood using magnetic beads coated with an anti-GPA antibody (FIG. 8B). As GPA is a marker of human RBCEVs but not mouse RBCEVs, we expected to purify the injected human RBCEVs, separating them from mouse EVs. RBCEVs were quantified based on FACS analysis of CFSE fluorescent signals from the magnetic beads. The analysis revealed that the self-peptide-ligated RBCEVs were much more abundant than control-peptide-ligated RBCEVs in the circulation of the injected mice (FIG. 8B). Moreover, we also injected DiR-labelled self-peptide-ligated RBCEVs in the tail vein and observed an enhanced biodistribution of the RBCEVs in multiple organs including the liver, spleen, lung, bone and kidneys (FIG. 8C). These data indicate that the conjugation with the self peptide can be used to increase the circulation and biodistribution of RBCEVs.

Conjugation of RBCEVs with biotrophic single domain antibodies requires a linker peptide

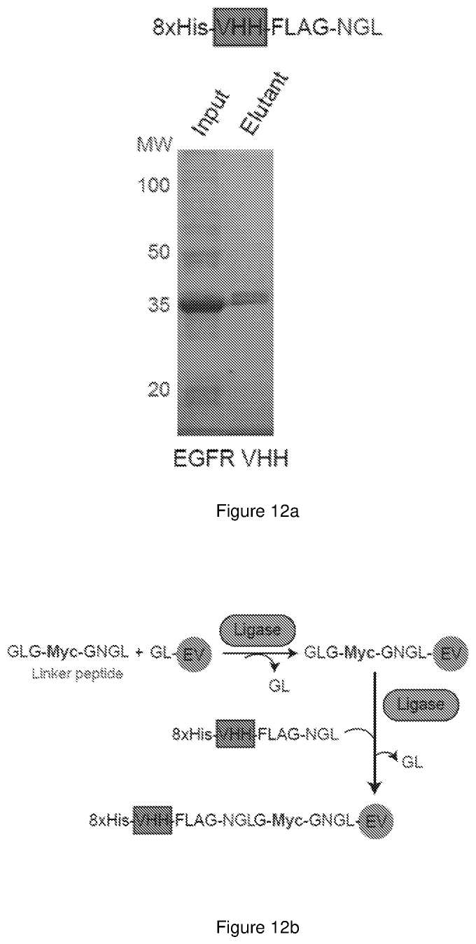

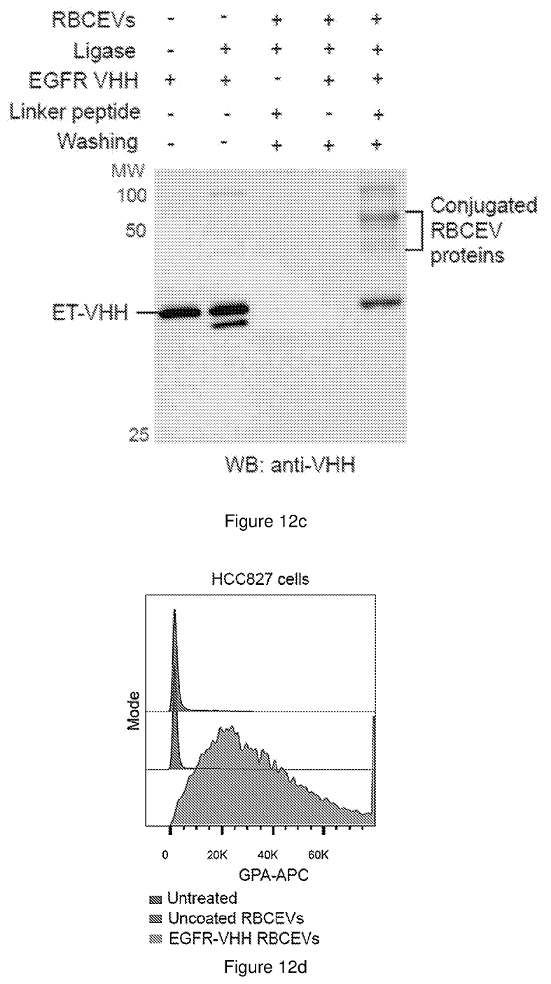

We sought to use sdAbs to guide the targeting delivery of RBCEVs because sdAbs are known for high specificity and ease of modification as they have only one polypeptide. In addition to the mCherry sdAb shown in FIG. 1, we produced another camelid sdAb (also called VHH) specific to EGFR with His tags, FLAG tag, HA tag and a ligase binding site (FIG. 12A). The purified EGFR VHH was approximately 37 kDa. This is a biotrophic antibody so it is larger than a typical sdAb. It has 2 high-affinity binding sites for EGFR.

After multiple failed attempt to ligate EGFR VHH to RBCEVs directly (probably due to the large size of the VHH), we designed a linker peptide to make a bridge between the VHH and RBCEVs (FIG. 12B). This linker peptide comprises of a Myc tag in the middle, a “GL” at the N terminus and a “NGL” at the C terminus. The “NGL” sequence facilitates the ligation of the peptide to RBCEVs. The “GL” sequence subsequently enables a ligation of the linker peptide to the VHH with “NGL”. We performed the ligation reaction with multiple controls. Using anti-VHH Western blotting, we observed the free VHH as a 37 kDa band (FIG. 12C). Addition of OaAEP1 ligase to the VHH resulted in 2 additional bands, probably due to possible cleavage and oligomerization of the VHH. RBCEVs were ligated to the VHH with or without an addition of the linker peptide and washed extensively using SEC and 4 rounds of centrifugation. Several proteins bands between 45 and 60 kDa were detected by anti-VHH antibody in the two-step VHH-ligation to RBCEVs that involved the linker peptide (FIG. 12C). These bands were different from those appeared due to the incubation of VHH with ligase only. No band was observed in the ligation of VHH with RBCEVs without the linker peptide. The data suggest that the ligation of EGFR VHH to RBCEVs required the addition of the linker peptide.

As a result of EGFR VHH conjugation, we observed increased binding of RBCEVs to EGFR-positive HCC827 cells compared to uncoated RBCEVs based on a FACS analysis of GPA on the surface of the cells (FIG. 12D).

Conjugation of RBCEVs with Single Domain Antibodies Promote Specific Uptake of RBCEVs by Target Cells

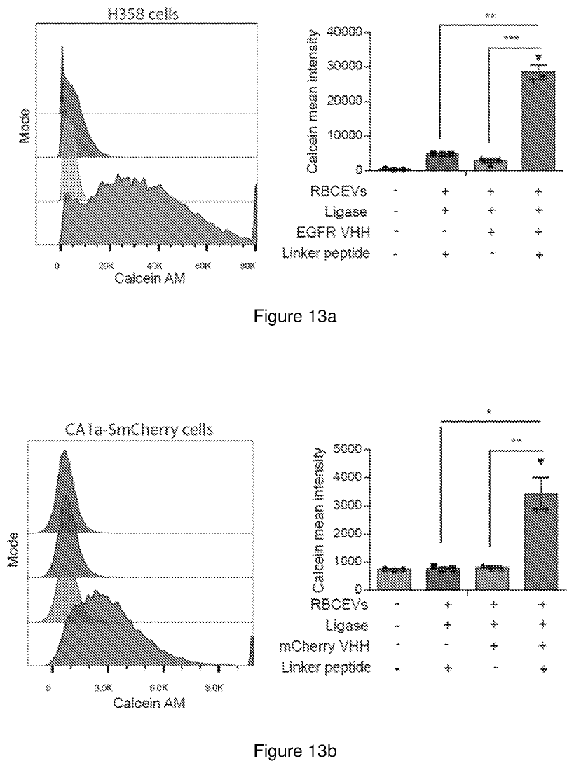

To test the effect of VHH conjugation on the uptake of RBCEVs, we labelled VHH-coated RBCEVs with Calcein AM and wash them using SEC. FACS analysis of Calcein AM showed that uptake of RBCEVs by H358 cells increased only when the RBCEVs were ligated in 2 steps with the linker peptide and EGFR targeting VHH (FIG. 13A). Similarly, we tested the ligation of mCherry-targeting VHH to RBCEVs and their uptake by CA1a cells with surface expression of mCherry protein. The uptake of RBCEVs by CA1a-SmCherry cells increased only with RBCEVs ligated to the linker peptide and mCherry VHH (FIG. 10B). Lack of linker peptide in the VHH ligation did not result in any enhanced uptake. Hence, the linker peptide is required for VHH ligation to RBCEVs.

Delivery of RNAs and Drugs using sdAb-Ligated RBCEVs

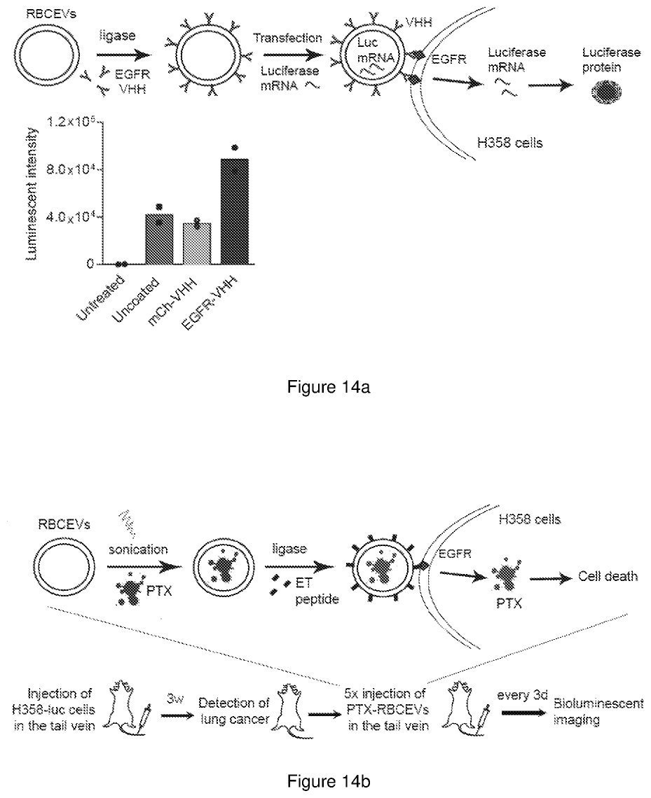

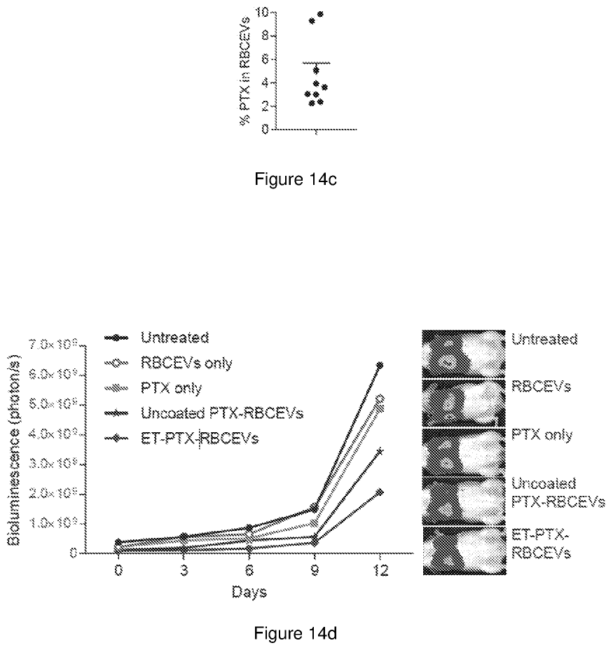

We have shown before that RBCEVs can be used to deliver ASOs, gRNAs or mRNAs to cancer cells. Here, we coupled of the ligation reaction and the RNA loading experiment. We found that RBCEVs need to be conjugated first, washed twice using centrifugation, and subsequently loaded with RNAs using EV-transfection reagents such as ExoFect (System Biosciences). Hence, we ligated EGFR VHH or mCherry VHH to RBCEVs and loaded them with a luciferase mRNA (FIG. 14A). H358 cells expressing EGFR but not mCherry were treated with these RBCEVs and luciferase activity and compared after 24 hours. RBCEVs ligated with EGFR VHH resulted in 2-fold higher luciferase activity in H358 cells than that after the treatment with uncoated RBCEVs or RBCEVs ligated with mCherry VHH albeit all the RBCEVs-treated cells showed higher luciferase signals than the untreated control (FIG. 14A). Therefore, RBCEVs were able to deliver luciferase mRNA to H358 cells with increased efficiency upon their conjugation with EGFR VHH.

We also optimized a protocol for loading paclitaxel (PTX), a chemotherapy drug commonly used for lung cancer treatments, into RBCEVs using sonication (FIG. 14B). Drug loaded RBCEVs were washed thoroughly and ligated with the EGFR-targeting peptide as described above. The modified RBCEVs were injected into NSG mice bearing H358 lung cancer every 3 days, at the same dose of PTX only that was used as a control. The concentration of PTX was determined using HPLC. On average ˜6% PTX was loaded into RBCEVs and unbound PTX was washed away (FIG. 14C). Bioluminescent imaging of the tumour showed that the EGFR-targeting RBCEVs enhanced the effect of PTX on tumour suppression compared to PTX only or uncoated PTX-loaded RBCEVs (FIG. 14D). These data suggest that targeted delivery of anti-cancer drug could increase the efficacy of the treatment by increasing the accumulation of the drug in the targeted tumour cells.

### Example 2

### METHODS

### Purification of EVs

Blood samples were obtained by Red Cross from healthy donors in Hong Kong with informed consents. All experiments with human blood samples were performed according to the guidelines and the approval of the City University of Hong Kong Human Subjects Ethics committee. RBCs were separated from plasma using centrifugation (1000×g for 8 min at 4° C.) and washed three time with PBS (1000×g for 8 min at 4° C.) and white blood cells were removed by using centrifugation and leukodepletion filters (Terumo Japan or Nigale, China). Isolated RBCs were collected in Nigale buffer (0.2 g/I citric acid, 1.5 g/I sodium citrate, 7.93 g/I glucose, 0.94 g/I sodium dihydrogen phosphate, 0.14 g/I adenine, 4.97 g/I sodium chloride, 14.57 g/I mannitol) and diluted 3 time in PBS containing 0.1 mg/ml Calcium Chloride and treated with 10 mM calcium ionophore (Sigma Aldrich) overnight (the final concentration of calcium ionophore was 10 μM). To purify EVs, RBCs and cell debris were removed by centrifugation at 600×g for 20 min, 1,600×g for 15 min, 3,260×g for 15 min and 10,000×g for 30 min at 4° C. The supernatants were passed through 0.45-μm-syringe filters. EVs were concentrated by using ultracentrifugation with a TY70Ti rotor (Beckman Coulter, USA) at 100,000×g or 50,000×g for 70 min at 4° C. EVs were resuspended in cold PBS. For labeling, half of the EVs were mixed with 20 pM PKH26 (Sigma Aldrich, USA). Labeled or unlabeled EVs were layered above 2 ml frozen 60% sucrose cushion and centrifuged at 100,000×g or 50,000×g for 16 hours at 4° C. using a SW41Ti rotor (Beckman Coulter) with reduced braking speed. The red layer of EVs (above the sucrose) was collected and washed once (unlabeled EVs) or twice (labeled EVs) with cold PBS using ultracentrifugation in a TY70Ti rotor (Beckman Coulter) at 100,000×g or 50,000×g for 70 min at 4° C. Of note, ultracentrifugation at 100,000×g was used for higher yield of RBCEVs. 50,000×g was used when we sought to treat EVs gently. All ultracentrifugation experiments were performed with a Beckman XE-90 ultracentrifuge (Beckman Coulter). Purified RBCEVs were stored in PBS containing 4% trehalose at −80° C. The concentration and size distribution of EVs were quantified using a NanoSight Tracking Analysis NS300 system (Malvern, UK). The protein contents of EVs were quantified using bicinchoninic acid assay (BCA assay). For transmission electron microscopy analysis of EVs, EVs were fixed on copper grids (200 mesh, coated with formvar carbon film) by adding equal amount of 4% paraformaldehyde. After washing with PBS, 4% uranyl acetate was added for chemical staining of EVs and images were captured using a Tecnai 12 BioTWIN transmission electron microscope (FEI/Philips, USA). The haemoglobin contents of RBCEVs were quantified using a haemoglobin quantification kit (Abcam).

Purification of Leukaemia EVs from THP1 Cells

THP1 cells were obtained from the American Type Culture Collection (ATCC, USA) and maintained in RPMI (Thermo Fisher Scientific) with 10% fetal bovine serum (Biosera, USA) and 1% penicillin/streptomycin (Thermo Fisher Scientific, USA). To make EV-free FBS, EVs were removed from FBS using ultracentrifugation at 110,000×g for 18 hours at 4° C. THP1 cells were cultured at 10⁶ cells/ml in the above medium with EV-free FBS and 0.2 μM calcium ionophore for 48 hours. Culture supernatants were collected from 5 flasks of treated THP1 cells. Cells and debris were removed by centrifugation at 300×g for 10 min, 400×g for 15 min, 900×g for 15 min at 4° C. The supernatant was further passed through a 0.45 μm filter, layered above 2 ml frozen 60% sucrose, and concentrated by using ultracentrifugation with a SW32 rotor at 100,000×g for 90 min at 4° C. EVs were collected from the interface and diluted 1:1 in cold PBS, and layered above 2 ml frozen 60% sucrose cushion in a SW41 rotor and centrifuged at 100,000×g for 12 hours at 4° C. (Beckman Coulter) with reduced braking speed. The red layer of EVs (above the sucrose) was collected and washed once (unlabeled EVs) or twice (labeled EVs) with cold PBS using ultracentrifugation in a TY70Ti rotor (Beckman Coulter) at 100,000×g for 70 min at 4° C. 500 pl EVs were collected from the interface and added to a qEV SEC column (Izon). 500 μl elutant was collected in each fraction. The concentration of EVs and protein were measured in 30 fractions using a Nanosight analyser and BCA assay. For ligation, the EVs from fraction 7 to 11 were combined and concentrated using centrifugation at 15,000μg for 20 minutes in an Amicon-15 filter with 100 kDa cut-off.

Peptide and sdAb Design

Biotinylated self peptide (Biotin-GNYTCEVTELTREGETIIELK-GGGGS-LPETGGG), Bi-YG20 peptide (Biotin-YHWYGYTPQNVIGLPETGGG, sortase binding site is underlined) and Biotin-TRNGL and other peptides listed in Table 1 were synthesized using 96/102 well automated peptide synthesizers and purified by high performance liquid chromatography (GL Biochem Ltd., Shanghai, China). The variable heavy chain (VHH) sequence of an anti-mCherry sdAb (387 bp) was obtained from Fridy et al⁹ with additional sortase binding site (LPETG) or a ligase binding site (NGL), a HA tag and a FLAG tag at the C terminus. A Myc tag, a thrombin cleavage site and 6 His tags were also added to the N-terminus of the VHH. The whole sequence of 6*His-SSG-thrombin-cleavage site-Myc-VHH-GSG-HA-GSG-LPETGGG-Flag (555 bp, 20 kDa, the italic font denotes the linkers) was synthesized and inserted into pET32(a+) plasmid, following a T7 promoter by Guangzhou IGE Biotechnology Ltd (China). The biotrophic EGFR-VHH sequence was obtained from Roovers et al (International journal of cancer, 2011, 129(8), 2013-2024) and cloned with 8 His tags, FLAG tag and a ligase-binding site in this order: 8*His-GSG-VHH-GSG-FLAG-NGL, into into pET32(a+) plasmid as described above.

TABLE 1
Sequences of peptides

| | | Peptide |
|---|---|---|
| | | sequence |
| | | from N to |
| Name | Abbreviation | C terminus |
| Scrambled | EL17 | GGGEQKLISE |
| control | | EDLG-NGL |
| peptide for | | |
| ligation | | |
| Biotinylated | TR5 | Biotin- |
| TR peptide | | TR-NGL |
| for | | |
| ligation | | |
| Biotinylated | YK16 | YHWYGYTPQNVI- |
| EGFR targeting | | GSGK-biotin |
| peptide | | |
| Biotinylated | YG20 | Biotin-YHWYGYTP |
| EGFR- | | QNVIGLPETGGG |
| targeting | | |
| peptide | | |
| for | | |
| sortagging | | |
| EGFR- | YG24 | YHWYGYTPQNVI- |
| targeting | | GGGGS-LPETGGG |
| peptide | | |
| for | | |
| sortagging | | |
| Linker | GL17 | GLGEQKLISEED |
| peptide | | LG-NGL |
| for | | |
| ligation | | |
| of VHH | | |
| Biotinylated | GG33 | Biotin-GNYTCE |
| self-peptide | | VTELTREGETII |
| for | | ELK-GGGGS- |
| sortagging | | LPETGGG |
| Self | GL29 | GNYTCEVTELTRE |
| peptide | | GETIIELK-GGGG |
| for ligation | | S-NGL |
| EGFR- | YL20 | YHWYGYTPQNVI- |
| targeting | | GGGGS-NGL |
| peptide for | | |
| ligation | | |
| BBCO-linker | GK-DBCO | GL-GSSGSGG- |
| peptide for | | DYKDDDDK-GG |
| ligation | | SGSGGK- |
| | | diarylcyclooctyne |
| | | (DBCO) |
| Azide-linker | Azide-GL | Azide-GSSGSGG- |
| peptide for | | EQKLISEEDL- |
| ligation | | GGSGGSGSG- |
| | | NGL |
| PEG-linker | ML | Maleimide- |
| peptide | | (PEG12)-NGL |
| for ligation | | |

Expression and Purification of Proteins

Competent BL21 (DE3) E.coli bacteria were transformed with pET30b-7M-SrtA plasmid (Addgene 51140) and spread on agar plates with kanamycin (Sigma), OaAEP1-Cys247Ala plasmid (provided by Dr. Bin Wu, Nanyang Technology University) or with pET32(a+)-VHH plasmid (cloned with specific VHH sequences) and spread on agar plate with Ampicillin, and incubate at 37° C. overnight. Single colonies were selected from each plate and culture in Lysogeny broth (LB) with shaking at 37° C. overnight. Protein expression was induced with 0.5 mM Isopropyl β-D-1-thiogalactopyranoside (IPTG) in LB at 25° C. for 16 h with shaking. The culture was collected and centrifuged at 6,000×g for 15 min at 4° C. The supernatant was removed and the pellet was resuspended in 50 mL binding buffer (500 mM NaCI, 25 mM Tris-HCI, 1 mM phenylmethane sulfonyl fluoride (PMSF), 5% glycerol) and transfer to a 50 mL centrifuge tube and centrifuge again.

Bacteria were lysed using a high pressure homogenizer (1000 psi) for 4-6 rounds. The cell lysate was centrifuge at 8000 rpm for 60 min at 4° C. The supernatant was collected and filtered through a 0.45 μm membrane (Millipore). The proteins were purified using the NGC-QUEST-10 fast protein liquid chromatography (FPLC) system (BioRad). Briefly, the sample was loaded into a 5-mL-Ni-charged cartridge (BioRad) equilibrated with the binding buffer. The column was washed with 3% elution buffer (500 mM NaCl, 25 mM Tris-HCl, 1 mM imidazole, 1 mM PMSF and 5% glycerol) and then eluted in 8% to 50% elution buffer. The flow rate was kept constant at 3 ml/min. Fractions of 2 ml was collected when the proteins appeared as UV280 peaks. The proteins were concentrated using a centrifugal filter (Millipore) and 4000×g centrifugation in a swinging-bucket rotor and filtered through a 0.22 μm membrane. The proteins were further purified using a HiLoad 16/600 Superdex 200 pg size exclusion chromatography column (GE Healthcare) with the FPLC system, in low ionic strength buffer (150 mM NaCl, 50 mM Tris-HCl), at 0.5 ml/min. The target protein was collected at the appropriate UV280 peak and confirmed using gel electrophoresis with Coomassie Blue staining. For OaAEP1 ligase, activation, a buffer comprised of 1 mM EDTA and 0.5 mM Tris 1 mM EDTA and 0.5 mM Tris (2-carboxyethyl) phosphine hydrochloride was added to the immature protein and the pH of the solution was adjusted to 4 with glacial acetic acid. The protein pool was incubated for 5 h at 37° C. Protein precipitation at this pH allowed removal of the bulk of the contaminating proteins by centrifugation. Activated proteins were concentrated by ultracentrifugation using a 10 kDa cutoff concentrator and stored at −80° C.

Sortagging EVs with Antibodies and Peptides Bearing LPETG Sequence

600 pmol sortase A was mixed with 2.75 pmol sdAb or 21 μmol peptides in 1× Sortase buffer (50 mM TrisHCl pH 7.5, 150 mM NaCl), and kept on ice for 30 min. Subsequently, 8×1011 EVs (˜50 μg EV proteins) were added into the sortase mixture, to a final concentration of 4 μM sortase A (˜10μg) and 20 μM VHH-LPETG (˜50 ˜g) in a total volume of 125 μl. The reaction was incubated at 4° C. for 60 mins with gentle agitation (20 rpm) on an end-over-end shaker. The conjugated EVs were added to 2 ml frozen 60% sucrose cushion and centrifuged at 100,000×g for 16 hours at 4° C. using a SW41Ti rotor (Beckman Coulter) with reduced braking speed. The red layer of EVs (above the sucrose) was collected and washed once with 16 ml cold PBS using ultracentrifugation in a 70Ti rotor (Beckman Coulter) at 100,000×g for 70 min at 4° C.

Coating EVs with Peptides Bearing TRNGL Sequence using OaAEP1 Cys247Ala Protein Ligase

Every 20 μl reaction mixture containing 3 μl RBCEVs (0.72×10¹¹ particles/ul, equivalent to 100 μg Haemoglobin in RBCEVs), 2.5 to 10 μl of 1 mM peptide and 5 μl of 10 μM ligase in PBS buffer, pH 7 to 7.4 (pH 7 is the optimal), to a final concentration of ligase (1 μM) and peptide (50 to 500 μM). Incubate the reaction at RT for 30 mins with gentle agitation (30 rpm) on end-over-end shaker. When the reaction was scaled up, longer incubation time was required e.g. 3 hours for ligation of 1-2 mg RBCEVs (based on Haemoglobin quantification).

Labelling Coated RBCEVs with Fluorescent Dyes

RBCEVs coated with peptides or sdAb were wash once with PBS by centrifugation at 21,000×g for 15 minutes at 4° C. Washed RBCEVs were incubated with 10 μM calcein AM for 20 minutes at room temperature or 20 μM CFSE for 1 hour at 37° C. or 2 μM DiR for 15 minutes at room temperature. The labelled RBCEVs were loaded immediately into a SEC column (Izon) and eluted with PBS. Fraction 7 to 10 (with pink-red color) were collected and wash 3 time by centrifugation at 21,000×g for 15 minutes at 4° C.

Loading RNAs and Drugs into RBCEVs

Ligated RBCEVs were washed with PBS at 21,000×g for 15 minutes at 4° C. 3 time before the loading of RNAs. 9 μg luciferase mRNA (Trilink) was loaded into 50 μg RBCEVs using a transfection reagent for 30 minutes. The EVs were then wash in PBS by centrifugation at 21,000×g for 3 time.

For drug loading, uncoated RBCEVs were incubated with 200 μg PTX in 1 ml PBS at 37 ° C. for 15 minutes. The mixture were sonicated using a Bioruptor (Biogenode) for 12 minutes at 4° C. then recovered at 3TC for 1 hour. The loaded RBCEVs were washed with PBS at 21,000×g for 15 minutes, quantified using the haemoglobin assay and coated with peptides as described above. The coated RBCEVs were repurified using SEC as described. To measure PTX loaded into RBCEVs, an aliquot of the loaded RBCEVs were centrifuge at 21,000×g for 15 minutes. The pellet was dried at 75 ° C. and resuspended in acetonitrile and centrifuged at 21,000×g for 10 minutes. The supernatant was passed through a 0.22 μm filter and analysed using HPLC.

Western Blot Analysis

Conjugated EVs were incubated with RIPA buffer supplemented with protease inhibitors (Biotool) for 5 min on ice. 30 μg of protein lysates were separated on 10% polyacrylamide gels and transferred to a Nitrocellulose membrane (GE Healthcare). PM5100 ExcelBand™ 3-color high range protein ladder (SmoBio, Taiwan) was loaded at 2 sides of the samples. Membranes were blocked with 5% non-fat milk in Tris buffered saline containing 0.1% Tween-20 (TBST) for 1 hour at room temperature and incubated with primary antibodies overnight at 4° C.: mouse anti-His/VHH (Genescript, dilution 1:1000), mouse anti-FLAG (Sigma, dilution 1:500). The blot was washed 3 times with TBST then incubated with HRP-conjugated anti-mouse secondary antibody (Jackson ImmunoResearch, dilution 1:10,000,) for 1 hour at room temperature. For biotinylated peptide detection, the blot was not incubated with any antibody but with HRP-conjugated streptavidin directly (Thermo Fisher, dilution 1:4000). The blot was imaged using an Azure Biosystems gel documentation system.

Treatment of Cancer Cells with Peptide or sdAb-Coated EVs

Human breast cancer SKBR3 cells, human lung cancer H358 and HCC827 cells were obtained from the American Type Culture Collection (ATCC, USA). Human breast cancer MCF10CA1a (CA1a) were obtained from Karmanos Cancer Institute (Wayne State University, USA). Acute myeloid leukemia MOLM13 and THP1 cells were obtained from DSMZ Collection of Microorganisms and Cell Cultures (Braunschweig, Germany). All the solid cancer and leukaemia cells were maintained in DMEM or RPMI (Thermo Fisher Scientific), respectively, with 10% fetal bovine serum (Biosera, USA) and 1% penicillin/streptomycin (Thermo Fisher Scientific, USA). To test the EV uptake, 100,000 SKBR3 cells were incubated with 6×10¹¹ PKH26-labeled uncoated or YG20-coated EVs in 500 μl growth medium per well in 24-well plates for 24 hours. In a shorter uptake assay, H358, HCC827, MOLM13 and THP1 cells were incubated with Calcein AM-labelled RBCEVs for 1 to 2 hours at 37° C. To identify the route of EV uptake, we added 25-100 μM EIPA, 5-20 μg/ml Filipin, 0.25-1 μM Wortmannin. In the EV binding assay, HCC827 cells were incubated with unlabelled RBCEVs for 1 hour at 4° C.

Flow Cytometry Analysis

RBCEVs treated SKBR3 or other cells were washed twice with PBS and resuspended in 100 μl FACS buffer (PBS containing 0.5% fetal bovine serum). The cells were incubated with 3 μl FITC-conjugated EGFR antibody (Biolegend) for 15 minutes on ice, in the dark, and wash twice with 1 ml FACS buffer. To quantify the peptide coating efficiency, 100 μg bi-YG20-coated or bi-TRNGL coated RBCEVs or uncoated RBCEVs (as a negative control) were incubated overnight with 2.5 μg latex beads (Thermo Fisher Scientific) at 4° C. on a shaker, washed three times with PBS and resuspended in 100 μl FACS buffer containing 1 μl streptavidin conjugated with Alexa Fluor 647 (AF647), incubated on ice for 15 minutes and washed twice with FACS buffer. Flow cytometry of latex beads or cells in FACS buffer was performed using a CytoFLEX-S cytometer (Beckman Coulter) and analyzed using Flowjo V10 (Flowjo, USA). The beads or cells were initially gated based on FSC-A and SSC-A to exclude the debris and dead cells (low FSC-A). The cells were further gated based on FSC-width vs. FSC-height, to exclude doublets and aggregates. Subsequently, the fluorescent-positive beads or cells were gated in the appropriate fluorescent channels: PE for PKH26, APC for AF647, as the populations that exhibited negligible signals in the unstained/untreated negative controls.

Generation of in Vivo Cancer Models and Treatments with RBCEVs

H358 cells were transduced with lentiviral vector (pLV-Fluc-mCherry-Puro) and selected with puromycin to create a stable cell line. 1 million H358-luc cells were injected into the tail vein of NSG mice (6-7 weeks old). After 3 weeks, bioluminescence in the lung was detected using IVIS Lumina II (Pekin Elmer) after an injection of D-luciferin. Mice with comparable bioluminescent signals were preconditioned with 0.5 to 5×109 human RBCs (1-7 days old after collection from the donors) or the ghosts of the same RBC numbers via a retro-orbital injection.

After 1 hour, for biodistribution experiment, the mice were injected with 100 μg DiR-labelled RBCEVs that were ligated with a control or EGFR-targeting peptide in the tail vein. After 8 hours, the mice were sacrificed and DiR fluorescence was measured immediately in the organs. For drug treatment, every 3 days, the mice were injected i.v. with 20 mg/kg paclitaxel (PTX) alone or an equivalent dose of PTX in RBCEVs with or without EGFR-peptide ligation, 1 hour after RBC preconditioning. The same amount of unloaded RBCEVs was used as a negative control.

Quantification of RBCEVs in the Circulation 500 μg CFSE-labelled peptide-ligated RBCEVs were injected into the tail vein of NSG mice. After 5 minutes, 100 μl blood was collected from the eye. Blood cells were removed and 20 μl plasma was incubated with 5 μl biotinylated GPA antibody for 2 hours at room temperature with gentle rotation. The mixture was then incubated with 20 μl streptavidin beads for 1 hour at room temperature. The beads were washed 3 times and resuspended in 500 μl FACS buffer for analysis of CFSE.

### REFERENCES

A number of publications are cited above in order to more fully describe and disclose the invention and the state of the art to which the invention pertains. Full citations for these references are provided below. The entirety of each of these references is incorporated herein.

1. Pitt, J. M., Kroemer, G. & Zitvogel, L. Extracellular vesicles: masters of intercellular communication and potential clinical interventions. J. Clin. Invest. 126,1139-1143 (2016).

2. Kamerkar, S. et al. Exosomes facilitate therapeutic targeting of oncogenic KRAS in pancreatic cancer. Nature 546,498-503 (2017).

3. Syn, N. L., Wang, L., Chow, E. K.-H., Lim, C. T. & Goh, B.-C. Exosomes in Cancer Nanomedicine and Immunotherapy: Prospects and Challenges. Trends Biotechnol. (2017). doi:10.1016/j.tibtech.2017.03.004

4. Usman, W. et al. Efficient RNA drug delivery using red blood cell extracellular vesicles. Nature Communications In press, (2018).

5. Vader, P., Breakefield, X. O. & Wood, M. J. A. Extracellular vesicles: emerging targets for cancer therapy. Trends in Molecular Medicine 20,385-393 (2014).

6. Kooijmans, S. A. A., Gitz-Francois, J. J. J. M., Schiffelers, R. M. & Vader, P. Recombinant phosphatidylserine-binding nanobodies for targeting of extracellular vesicles to tumor cells: a plug-and-play approach. Nanoscale 10,2413-2426 (2018).

7. Shi, J. et al. Engineered red blood cells as carriers for systemic delivery of a wide array of functional probes. Proc. Natl. Acad. Sci. U.S.A. 111,10131-10136 (2014).

8. Pishesha, N. et al. Engineered erythrocytes covalently linked to antigenic peptides can protect against autoimmune disease. Proc. Natl. Acad. Sci. U.S.A. 114,3157-3162 (2017).

9. Fridy, P. C. et al. A robust pipeline for rapid production of versatile nanobody repertoires. Nat Methods 11,1253-1260 (2014).

10. Rodriguez, P. L. et al. Minimal ‘Self’ Peptides That Inhibit Phagocytic Clearance and Enhance Delivery of Nanoparticles. Science 339, 971-975 (2013).

11. Li, Z. et al. Identification and characterization of a novel peptide ligand of epidermal growth factor receptor for targeted delivery of therapeutics. FASEB J. 19, 1978-1985 (2005).

12. Yang, R. et al. Engineering a Catalytically Efficient Recombinant Protein Ligase. J. Am. Chem. Soc. 139,5351-5358 (2017).

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

1. An extracellular vesicle comprising an exogenous polypeptide tag, wherein the tag is covalently linked to a membrane protein of the extracellular vesicle.

2. The extracellular vesicle according claim 1, wherein the tag comprises one or more functional domain(s) wherein the functional domain is capable of binding to a target moiety, capable of being detected and/or capable of inducing a therapeutic effect.

3. The extracellular vesicle according to claim 2, wherein the functional domain comprises an antibody or antigen binding fragment, preferably a sdAb.

4. The extracellular vesicle according to any one of the preceding claims, wherein the extracellular vesicle is a microvesicle or exosome, preferably a microvesicle.

5. The extracellular vesicle according to claim 4, wherein the extracellular vesicle is a microvesicle derived from a red blood cell.

6. The extracellular vesicle according to any one of the preceding claims, wherein the extracellular vesicle is loaded with a cargo.

7. The extracellular vesicle according to claim 6, wherein the cargo is a nucleic acid, peptide, protein or small molecule.

8. The extracellular vesicle according to claim 7, wherein the cargo is a nucleic acid selected from the group consisting of an antisense oligonucleotide, a messenger RNA, a long RNA, a siRNA, a miRNA, a gRNA or a plasmid.

9. A composition comprising one or more extracellular vesicles according to any one of claims 1-8.

10. An extracellular vesicle or composition according to any one of the preceding claims, for use in a method of treatment.

11. A method of treatment, the method comprising administering an extracellular vesicle according to claim 1 to a patient in need of treatment.

12. Use of an extracellular vesicle or composition according to any one of claims 1-11 in the manufacture of a medicament for the treatment of a disease or disorder.

13. The extracellular vesicle or composition for use, method of treatment or use according to any one of claims 10-12 wherein the method of treatment involves administration go an extracellular vesicle or composition according to any one of claims 1-9 to a subject with a genetic disorder, inflammatory disease, cancer, autoimmune disorder, cardiovascular disease or a gastrointestinal disease.

14. The extracellular vesicle or composition for use, method of treatment or use according to claim 13 wherein the subject has cancer, the cancer optionally selected from leukemia, lymphoma, myeloma, breast cancer, lung cancer, liver cancer, colorectal cancer, nasopharyngeal cancer, kidney cancer or glioma.

15. A method comprising contacting an extracellular vesicle with a tag and a protein ligase under conditions which allow covalent binding between the tag and a surface protein of the extracellular vesicle, thereby generating a tagged extracellular vesicle.

16. A method comprising:
(a) contacting an extracellular vesicle with a peptide and first protein ligase under conditions which allow covalent binding between the peptide and a surface protein of the extracellular vesicle, thereby generating a peptide tagged extracellular vesicle; and
(b) contacting the peptide tagged extracellular vesicle with a functional domain peptide and a second protein ligase under conditions which allow covalent binding between the peptide covalently bound to the extracellular vesicle and the functional domain peptide.

17. The method according to claim 16 wherein the first and second peptide ligases are the same.

18. The method according to claim 16 wherein the first and second peptide ligases are different.

19. The method of claim 15 or claim 16 wherein the method further comprises contacting the extracellular vesicle with a cargo and electroporating to encapsulate the cargo with the extracellular vesicle.

20. The method according to any one of claims 16 to 18 wherein the protein ligase is selected from the group consisting of a sortase or AEP1, preferably sortase A.

21. An extracellular vesicle obtained by a method according to any one of claims 15-20.

22. A tag, the tag comprising a binding molecule and a protein ligase recognition site, the tag optionally further comprising a spacer, the spacer arranged between the binding molecule and the protein ligase recognition site.

23. Nucleic acid encoding the tag according to claim 22.
